(19) United States
(12) Patent Application Publication
(10) Pub. No.: US 2026/0139260 A1
(43) Pub. Date: May 21, 2026

(54) TRANSCRIPTIONAL REGULATORS AND POLYNUCLEOTIDES ENCODING THE SAME

(71) Applicant: Novozymes A/S, Bagsvaerd (DK)

(72) Inventors: Amanda J. Fischer, Davis, CA (US); Katie Chan, San Mateo, CA (US); Ping Yan, Davis, CA (US)

(73) Assignee: Novozymes A/S, Bagsvaerd (DK)

(21) Appl. No.: 18/564,304

(22) PCT Filed: May 20, 2022
(86) PCT No.: PCT/US2022/030222

§ 371 Date: Nov. 27, 2023

Related U.S. Application Data
(60) Provisional application No. 63/193,876, filed on May 27, 2021.

(51) Int. Cl. C12N 15/81; C12N 1/16; C12N 9/24; C12R 1/885

(52) U.S. Cl. CPC C12N 15/81; C12N 1/16; C12N 9/2497; C12N 2830/34; C12R 2001/885

(57) ABSTRACT

The present invention relates to transcriptional regulator polypeptides, polynucleotides encoding the transcriptional regulator polypeptides, and to nucleic acid constructs, vectors and host cells comprising the polynucleotides as well as methods of producing a polypeptide of interest in host cells overexpressing the transcriptional regulators, to methods of increasing the oxygen uptake rate and/or decreasing viscosity of the culture broth in a fungal host cell cultivation process, to methods of producing a transcriptional regulator polypeptide, to the use of a transcriptional regulator polypeptide, and to the production of fungal biomass.

### REFERENCE TO A SEQUENCE LISTING

This application contains a Sequence Listing in computer readable form named Corrected2_SQ.txt (created on Jul. 21, 2025, containing 156,206 bytes), which is incorporated herein by reference.

### BACKGROUND OF THE INVENTION

### Field of the Invention

The present invention relates to transcriptional regulator polypeptides, polynucleotides encoding the transcriptional regulator polypeptides, and to nucleic acid constructs, vectors and host cells comprising the polynucleotides as well as methods of producing a polypeptide of interest in host cells overexpressing the transcriptional regulators, to methods of increasing the oxygen uptake rate and/or decreasing viscosity of the culture broth in a fungal host cell cultivation process, to methods of producing a transcriptional regulator polypeptide, to the use of a transcriptional regulator polypeptide, and to the production of fungal biomass.

### Description of the Related Art

Recombinant fungal host cells such as Trichoderma reesei are widely applied in the industry due to their excellent capability of secreting large quantities of cellulases and other proteins. Recombinant proteins produced in fungal host cell systems are often valuable proteins, such as recombinantly produced glucoamylases with host cells and production methods as described in WO2011127802. For industrial and commercial purposes, the productivity of the applied cell systems, i.e. the production of total protein per fermentation unit, is an important factor of production costs. Other favorable factors are a low culture broth viscosity, decreased biomass generation in relation to product formation, and increased oxygen uptake rates allowing increased product formation. Traditionally, yield increases have been achieved through mutagenesis and screening for increased production of proteins of interest. However, this approach is mainly only useful for the overproduction of endogenous proteins in isolates containing the enzymes of interest. Therefore, for each new protein or enzyme product, a lengthy strain and process development program is required to achieve improved productivities.

For the overexpression of heterologous proteins in fungal host cell systems, the production process is recognized as a complex multi-phase and multi-component process. Cell growth and product formation are determined by a wide range of parameters, including the composition of the culture medium, culture broth viscosity, fermentation pH, temperature, dissolved oxygen tension, shear stress, and fungal morphology. Generally, oxygen transfer and oxygen uptake are influenced by the presence of cells in fermentation broths. The effect depends on the morphology of the organism and the cell concentration. Cells with complex morphology, such as branched hyphae of fungal cells, generally lead to lower oxygen transfer and uptake rates by interfering with bubble break-up and promoting coalescence (P. M. Doran, Bioprocess Engineering Principles, 2nd Edition, Academic Press, 2013). Also, higher levels of culture broth viscosity are associated with reduced oxygen transfer to the cultured cells which can be counteracted by maintaining a pellet suspension medium in special gas-lift bioreactors (M. Moo-Young et al., Biotechnol. Bioeng., 30 (1987), pp. 746-753). Meeting the host cell's metabolic demand for oxygen supply is a key factor for high level protein production, both for recombinant protein production and/or native host cell protein production. The rheological properties of culture broths strongly affect fermentation performance, especially if aerobic microorganisms are employed. Very high broth viscosity is encountered in some fermentation systems such as filamentous fungal host cells. The high broth viscosity presents serious problems to mixing, heat supply, and oxygen transfer. These problems, in turn, limit the production capacities and efficiencies of the fermentation processes. For example, the volumetric oxygen transfer coefficient, kLa, in penicillin fermentations has been shown to decrease as the broth viscosity increases with cell growth (L.-K. Ju et al., Biotechnol. Bioeng. 38 (1991) 1223).

Various approaches to improve expression and secretion have been used in fungi. For the expression of heterologous genes, codon-optimized, synthetic genes can improve the transcription rate, whereas the overexpression of secretion chaperones is used to protect the heterologous protein from degrading. To obtain high-level expression of a particular gene, a well-established procedure is targeting multiple copies of the recombinant gene constructs to the locus of a highly expressed endogenous gene. A further strategy for improving protein yield is described in WO2011/075677 (Novozymes A/S) by the disruption of native proteases. Also, extensive efforts are ongoing for understanding the intricate regulatory network controlling endogenous fungal gene expression, including the design of synthetic promoters and synthetic expression systems as described in WO2017144777.

Despite the discussed approaches, it is of continuous interest to further improve recombinant protein production in fungal host cells. The object of the present invention is to provide a modified host strain and a method of protein production with increased protein productivity and/or with favorable cultivation characteristics.

### SUMMARY OF THE INVENTION

The present invention is based on the surprising and inventive finding that the overexpression of a fungal transcriptional regulator polypeptide yields in increased host cell protein production and secretion, as well as in increased production and secretion of recombinant proteins of interest. Furthermore, the inventors have surprisingly found that the overexpression of the fungal transcriptional regulator polypeptide resulted in a culture broth with increased oxygen uptake rate and/or reduced viscosity when cultivated in aerobic fermentation conditions. The identified regulator polypeptides are used in a method of production of recombinant polypeptides and/or host cell protein in fungal host cells, such as Trichoderma host cells but may also be applied for in-vitro transcription regulation, such as in cell-free systems or other protein expression platforms. Furthermore, the identified regulator polypeptides are also used in a method of protein production with increased oxygen uptake rate and/or reduced viscosity of the generated culture broth. Novel polynucleotides encoding the transcriptional regulator polypeptides, and a method of producing heterologous and native proteins using said polynucleotides are described.

As described in the Examples, the inventors have identified that overexpression of the fungal transcriptional regulator polypeptide surprisingly resulted in increased yield and/or secretion of total host cell protein and different classes of proteins of interest. Therefore, we expect that these findings also apply for other proteins of interest, such as other glycoproteins, and in particular to other heterologous proteins. Additionally, it was also totally unexpected that the overexpression of the regulator polypeptide thereof resulted in increased oxygen uptake rates and/or decreased culture broth viscosity which is favors a high yield of protein per biomass. A further unexpected observing was that overexpression of said regulator polypeptide lead to increased fungal biomass formation. Sequence analysis of the amino acid sequence of the transcriptional regulator polypeptide revealed that the polypeptide comprises at least one zinc finger domain which represent DNA-binding motifs of the polypeptide. Said at least one domain being postulated to significantly contribute to the positive effects in cultivation and protein expression described above.

Thus, in a first aspect the present invention relates to a fungal host cell comprising in its genome at least one first heterologous promoter operably linked to a first polynucleotide encoding a fungal transcription regulator polypeptide, or variant thereof, comprising or consisting of an amino acid sequence having a sequence identity of at least 60% to SEQ ID NO:24.

The presence of the first polynucleotide results in an increased level of the fungal transcriptional regulator polypeptide, or variant thereof, in the fungal host cell, relative to an isogenic or parent fungal host cell which lacks said first heterologous promoter operably linked to the first polynucleotide. The increased expression of the regulator polypeptide or variant thereof encoded by the first polynucleotide favors increased host cell protein production and/or secretion, increased protein of interest production and/or secretion, reduced culture broth viscosity, increased total feed supplementation and/or increased culture broth oxygen uptake rate, relative to an isogenic or parent fungal host cell which lacks said first heterologous promoter operably linked to the first polynucleotide, when cultivated under aerobic fermentation conditions.

In a second aspect, the present invention relates to a method for producing at least one polypeptide of interest, the method comprising:
i) providing a fungal host cell according to the first aspect,
ii) cultivating said fungal host cell under conditions conducive for expression of the at least one polypeptide of interest; and
iii) optionally, recovering the at least one polypeptide of interest.

Utilizing the host cell of the first aspect in the production method of said second aspect, at least one recombinant protein of interest and/or at least one native host cell protein of interest can be expressed and secreted in said host cells with increased yield relative to an isogenic or parent fungal host cell which lacks said first heterologous promoter operably linked to the first polynucleotide. The method also allows for the simultaneous expression and secretion of two or more proteins of interest, such as three, four or more proteins of interest. Furthermore, the method allows a fermentation at low culture broth viscosity levels, resulting in an increased oxygen uptake rate, presumably being one of the factors allowing the increased protein production and/or secretion by the host cells of the invention.

In a third aspect, the present invention relates to a nucleic acid construct comprising at least one first heterologous promoter operably linked to a first polynucleotide encoding a fungal transcriptional regulator polypeptide, or variant thereof, comprising or consisting of an amino acid sequence having a sequence identity of at least 60% to SEQ ID NO:24.

In a fourth aspect, the present invention relates to an expression vector comprising a nucleic acid construct according to the third aspect.

In a fifth aspect, the present invention relates to a method for generating a recombinant fungal host cell with increased protein secretion relative to an isogenic or parent fungal host cell which lacks said first heterologous promoter operably linked to the first polynucleotide, the method comprising:
i) providing a fungal host cell secreting at least one protein,
ii) providing at least one nucleic acid construct or the at least one expression vector according to the third and/or fourth aspect, respectively, and
iii) integrating the at least one nucleic acid construct or the at least one expression vector into the genome of the host cell, wherein the at least one nucleic acid construct or the at least one expression vector confers an increased level of the transcriptional regulator polypeptide, or variant thereof, to the recombinant host cell relative to an isogenic cell lacking said nucleic acid construct or expression vector.

In a sixth aspect, the present invention relates to a method for aerobic cultivation of recombinant fungal host cells, the method comprising:
i) providing a recombinant fungal host cell according to the first aspect, or a recombinant fungal host cell generated by the method of the fifth aspect,
ii) cultivating the recombinant fungal host cell under aerobic conditions conducive for expression of the at least one polypeptide of interest,
   wherein the aerobic cultivation of the recombinant fungal host cell is characterized by the formation of a culture broth with an increased oxygen uptake rate and/or a reduced viscosity, relative to the oxygen uptake rate and/or viscosity of a culture broth generated by the cultivation of an isogenic fungal host cell lacking the at least one nucleic acid construct and/or the at least one expression vector, when cultivated under identical conditions.

In a seventh aspect, the present invention relates to methods for producing at least one transcriptional regulator polypeptide, the method comprising:
i) providing a fungal host cell according to the first aspect,
ii) cultivating said fungal host cell under conditions conducive for expression of the at least one transcriptional regulator; and
iii) optionally, recovering the at least one transcriptional regulator.

In an eighth aspect, the invention also relates to the use of the transcriptional regulator polypeptide for in-vitro transcription regulation, wherein the transcriptional regulator polypeptide is expressed by a fungal cell according to the first aspect, or wherein the transcriptional regulator polypeptide is produced by the method according to the seventh aspect. The use of the transcriptional regulator polypeptide is particularly advantageous in protein production in cell-free systems and other in-vitro expression systems.

In a ninth and final aspect, the invention relates to a method for producing fungal biomass, the method comprising:
i) providing a fungal host cell according to any one of the first aspect,
ii) cultivating said fungal host cell under conditions conducive for expression of the transcriptional regulator polypeptide; and optionally
iii) recovering the fungal host cells.

### Definitions

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter perse. For example, description referring to “about X” includes the aspect “X”.

Unless defined otherwise or clearly indicated by context, all technical and scientific terms used herein have the same meaning as commonly understood by one of ordinary skill in the art to which this invention belongs.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Constitutive promoter: The term “constitutive promoter” means an unregulated promoter that allows for continual transcription of its associated gene. The term “semi-constitutive promoter” means a partly regulated promoter that allows for transcription of its associated gene depending on e.g. cell cycle stage or extracellular factors, such as cultivation conditions. The term “inducible promoter” means a promoter which allows for transcription of its associated gene upon the presence of one or more inducer molecules, and which decreases/prevents the transcription of the associated gene in the absence of the inducer molecule(s).

Control sequences: The term “control sequences” means nucleic acid sequences necessary for expression of a polynucleotide encoding a polypeptide of the present invention. Each control sequence may be synthetic, native (i.e., from the same gene) or heterologous (i.e., from a different gene) to the polynucleotide encoding the polypeptide or native or heterologous to each other. Such control sequences include, but are not limited to, a leader peptide, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

DNA binding motif: The term “DNA-binding motif” means an amino acid sequence of a polypeptide which is configured to bind to a specific DNA sequence. DNA-binding motifs particularly include zinc finger domains, which are relatively small protein motifs containing one or more finger-like protrusion configured to make tandem contacts with their target DNA sequence. The binding properties of the zinc finger domain depend on the amino acid sequence of the domains, whereas zinc finger-containing proteins often are involved in the regulation of gene transcription, protein translation, mRNA trafficking, cell adhesion, and protein folding. One class of zinc fingers are the so-called “C₂H₂” or “Cys₂His₂-like” zinc fingers which adopt a simple beta-beta-alpha fold and have the amino acid sequence motif: X₂—Cys-X_{2,4}-Cys-X₁₂-His-X_{3,4,5}-His. Cys₂His₂-like zinc fingers often occur as tandem repeats with two, three or more fingers comprising the DNA-binding domain of the protein. These tandem arrays can bind in the major groove of DNA and are typically spaced at 3-bp intervals. The α-helix of each domain enables sequence-specific contacts to DNA bases; residues from a single recognition helix can contact four or more bases to yield an overlapping pattern of contacts with adjacent zinc fingers. A non-limiting example for a DNA binding motif of a polypeptide are the motifs of SEQ ID NO:79 and SEQ ID NO:80, comprised as amino acids corresponding to amino acids 257-281 and amino acids 286-311 in the transcriptional regulator polypeptide of SEQ ID NO:24, respectively.

Endogenous: The term “endogenous” means, with respect to a host cell, that a polypeptide or nucleic acid does naturally occur in the host cell, meaning that a transcriptional regulator which is endogenous to a host cell occurs naturally in said host cell and is native in said cell. As non-limiting examples, the polypeptide with the amino acid sequence of SEQ ID NO:24 and the promoter with the nucleic acid sequence of SEQ ID NO:3 each are endogenous to Trichoderma reesei host cells.

Expression: The term “expression” means any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fungal biomass/fungal cell: In the context of the present invention, the term “fungal cell” also includes hyphae and other cellular structures. “Fungal cell” as used herein thus includes fungal biomass in general. Typically, fungal biomass is measured as dry weight or wet weight of a plurality of fungal cells.

Glycomacropeptide: The term “Glycomacropeptide” or “GMP” means a natural protein found in sweet cheese whey. GMP is uniquely suited to the PKU diet because it is the only known dietary protein that contains no phenylalanine residues in its pure form.

Glycoprotein: The term “glycoprotein” means a conjugated protein in which the non-protein group is a carbohydrate. Glycoproteins contain oligosaccharide chains/glycans covalently attached to polypeptide sidechains. The carbohydrate is attached to the protein during co-translational modification and/or post-translational modification. Glycoproteins can contain N-linked and/or O-linked oligosaccharide residues. Non-limiting examples for a glycoprotein are a cellobiohydrolase, such as the cellobiohydrolase I of SEQ ID NO: 78, an amyloglucosidase, such as the amyloglucosidase of SEQ ID NO: 76, and a beta-mannosidase, such as the beta-mannosidase of SEQ ID NO: 77.

Glycosylase: The term “glycosylase” means a protein with glycosylase activity (EC number 3.2). Non-limiting examples for glycosylases are (i) an amyloglucosidase (EC number 3.2.1.3) that catalyzes the hydrolysis of terminal (1->4)-linked alpha-D-glucose residues successively from non-reducing ends of the chains with release of beta-D-glucose, (ii) a cellobiohydrolase such as a cellobiohydrolase I (CBH I) or cellobiohydrolase II (CBH II) (EC number 3.2.1.91), and (iii) a mannosidase such as a beta-mannosidase (EC number 3.2.1.25).

For purposes of the present invention, glucoamylase activity, CBH I activity, and beta-mannosidase activity is determined according to the procedure described in the Examples. The term “glucoamylase” is interchangeable with the terms “amyloglucosidase”, “glucan 1,4-α-glucosidase”, and/or “γ-amylase”. The term “beta-mannosidase” is interchangeable with the terms “beta-d-mannosidase”, “beta-man”, “man2a”, “mannase”, “hvbii”, “cmman5a”, “beta-d-mannoside mannohydrolase”, “beta-mannoside mannohydrolase”, and “beta-mannosidase 2a”. The term “cellobiohydrolase” is interchangeable with the terms “1,4-beta-cellobiohydrolase”, “1,4-beta-D-cellobiohydrolase”, “avicelase”, and “CBH”.

Heme-containing polypeptide: The term “heme-containing polypeptide” means a polypeptide which has a heme incorporated. The term “heme” means an iron-containing compound of the porphyrin class which forms the non-protein part of e.g. hemoglobin and other heme-containing polypeptides. Non-limiting examples for heme-containing polypeptides are proteins which give a meat-like flavor and/or a meat like color when added to a food or feed product, such as hemoglobin, peroxygenases or peroxidases. Non-limiting examples for heme-containing polypeptides are active or inactivated heme-containing enzymes selected from the list of polypeptides with at least 80% sequence identity to the polypeptides with SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, and SEQ ID NO:97.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, or domain of a polypeptide or nucleic acid is not naturally associated with the polypeptide or nucleic acid. As a non-limiting example, with respect to a polypeptide or nucleic acid, a heterologous promoter operably linked to a polynucleotide encoding the polypeptide of SEQ ID NO:24 is a promoter sequence natively associated with the regulation of the expression of a gene other than the gene encoding the mature polypeptide of SEQ ID NO:24. As a non-limiting example, with respect to a polypeptide or nucleic acid, a heterologous promoter may be a synthetic promoter controlling the expression of the transcriptional regulator, and/or controlling the expression of the at least one polypeptide of interest.

Host cell: The term “host cell” means any microbial, fungal or plant cell into which a nucleic acid construct or expression vector comprising a polynucleotide of the present invention has been introduced. Methods for introduction include but are not limited to protoplast fusion, transfection, transformation, electroporation, conjugation, and transduction. In some embodiments, the host cell is an isolated recombinant host cell that is partially or completely separated from at least one other component with, including but not limited to, proteins, nucleic acids, cells, etc.

Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 55° C. Medium-high stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 60° C. High stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 65° C. Very high stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 70° C.

Isogenic cell: The term “isogenic” refers, with respect to a host cell, to a parent or clonal host cell with an essentially identical genotype, e.g. a parent host cell having essentially identical background mutations as the daughter cell, yet with specific differences due to a later on introduced additional mutation or polynucleotide to the daughter cell resulting in a daughter cell with the additional mutation and/or polynucleotide but the daughter cell otherwise being isogenic to the parent cell.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Mature polypeptide: The term “mature polypeptide” means a polypeptide in its mature form following N-terminal processing (e.g., complete or partial removal of signal peptide and/or leader peptide). In one aspect, the mature polypeptide comprises one of SEQ ID NO: 24, SEQ ID NO: 76, SEQ ID NO: 77 and SEQ ID NO: 78.

Mature polypeptide coding sequence: The term “mature polypeptide coding sequence” means a polynucleotide that encodes a mature polypeptide having biological activity. In one aspect, the mature polypeptide coding sequence is nucleotides 1 to 1160 of SEQ ID NO:25 or nucleotides 1 to 1092 of SEQ ID NO:26.

Native: The term “native” means a nucleic acid or polypeptide naturally occurring in a host cell.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Oxygen uptake rate: The term “oxygen uptake rate” or “OUR” means a rate at which the biomass absorbs the available oxygen in the shake flask or bioreactor. OUR is calculated as follows:

$$OUR = {k}_{L} a ( {[ {O}_{2} ]}^{⋆} - [ {O}_{2} ] ) - d [ {O}_{2} ] / dt$$

where k_{L}a is the volumetric mass-transfer coefficient for the respective shake flask or bioreactor in the respective operating conditions, and [O₂]* is the saturation concentration. d [O₂]/d t (=OTR, oxygen transfer rate) is used to calculate the oxygen consumption during a certain time period. In order to calculate the OUR of the cells or microorganisms the k_{L}a has to be known, estimated, or determined. The oxygen content inside the shake flasks or bioreactor results from oxygen consumption by the cells or microorganisms inside the culture media and continuous oxygen transfer from outside into the media. In order to determine OUR of the cells or microorganisms the oxygen transfer into the shake flask or bioreactor has to be taken into account. The OUR can indirectly be assessed, wherein an increased oxygen consumption, increased oxygen feed, and/or increased total feed is associated with an increased OUR. Oxygen transfer through microbial cells controls the most of aerated fermentation systems. The amount of dissolved oxygen into the broths is limited by its solubility and mass transfer rate, as well as by its consumption rate on cells metabolic pathways, cell morphology, and increased culture broth viscosity.

The k_{L}a (Volumetric Mass Transfer Coefficient) and the OTR (Oxygen Transfer Rate) detail how efficient oxygen is transferred from the gas bubbles into the bioreactor medium, i.e. how much oxygen is available for the cultivated biomass. The rate at which the biomass absorbs the available oxygen is described using the OUR (Oxygen Uptake Rate). The OTR is defined by the k_{L}a and the difference between the oxygen concentration of the introduced gas and the oxygen concentration in the medium:

$$OTR = {k}_{L} a ( {C}^{⋆} - {C}_{L} )$$

$$OTR [ mg {O}_{2} / L / h ]$$

k_{L}: oxygen transfer coefficient (cm/h)
a: gas-liquid interfacial area per unit vol. (cm²/cm³)
C*: saturation oxygen concentration in media (mmol/L)
C_{L}: actual oxygen concentration in the media (=DO) (mmol/L)

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the no-brief option must be specified in the command line. The output of Needle labeled “longest identity” is calculated as follows:

$$( Identical Residues \times 100 ) / ( Length of Alignment - Total Number of Gaps in Alignment )$$

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

$$( Identical Deoxyribonucleotides \times 100 ) / ( Length of Alignment - Total Number of Gaps in Alignment )$$

Therapeutic polypeptide: The term “therapeutic polypeptide” means any polypeptide or protein, or variant thereof, which is suitable for use in the therapy of human diseases or conditions, or for use in veterinary medicine. Non-limiting examples for therapeutic polypeptides are antibody-based drugs, Fc fusion proteins, an anticoagulant, a blood factors, a bone morphogenetic protein, an engineered protein scaffold, an enzyme, a growth factor, a hormone, an interferon (e.g. an interferon alpha-2b), an interleukin, a lactoferrin, an alpha-lactalbumin, a beta-lactalbumin, an ovomucoid, an ovostatin, a cytokine, an obestatin, a human galactosidase (e.g. human alpha-galactosidase A) and a thrombolytic.

Transcriptional regulator polypeptide: The term “transcriptional regulator polypeptide” is interchangeable with the terms “transcription factor” or “TF” or “regulator polypeptide” and means a DNA-binding polypeptide that controls the rate of the transcription of genetic information from DNA to mRNA by binding to a specific polynucleotide sequence. Transcriptional regulator polypeptides function alone and/or together with one or more other polypeptides or transcription factors in a complex by promoting or blocking the recruitment of RNA polymerase. Regulator polypeptides are characterized by comprising at least one DNA-binding domain which often attaches to a specific DNA sequence adjacent to the genetic elements which are regulated by the transcriptional regulator polypeptide. The binding to the DNA sequence may take place via one or more zinc finger domains of the transcriptional regulator polypeptide. A transcriptional regulator may regulate the expression of a protein of interest either directly, i.e. by activating the transcription of the gene encoding the protein of interest by binding to its promoter, or indirectly, i.e. by activating the transcription of a further transcription factor which regulates the transcription of the gene encoding the protein of interest by binding to the promoter of the further transcription factor. Non-limiting examples for fungal transcriptional regulator polypeptides are the polypeptides encoded by the polynucleotide with SEQ ID NO:25, such as the regulator polypeptide with SEQ ID NO:24 or variants thereof. A non-limiting example for direct expression regulation by the fungal transcriptional regulators is the direct regulation of cbh1 gene expression by the regulator polypeptide with SEQ ID NO:24 or variants thereof. A non-limiting example for a further transcription factor, whose expression can be regulated by the transcriptional regulator polypeptide or variants thereof, is the xylanase regulator 1 (xyr1). The xylanase regulator 1 polypeptide is a regulator polypeptide which induces the expression of xylanase.

Variant: The term “variant” means a transcriptional regulator polypeptide having at least one DNA-binding domain for binding on at least one specific (genomic) polynucleotide binding sequence, the polypeptide variant comprising a man-made mutation, i.e., a substitution, insertion, and/or deletion (e.g., truncation), at one or more (e.g., several) positions to alter the expression of at least one gene sequence adjacent to the binding sequence, such as increasing the expression of at least one gene sequence by promoting the recruitment of RNA polymerases. A substitution means replacement of the amino acid occupying a position with a different amino acid; a deletion means removal of the amino acid occupying a position; and an insertion means adding an amino acid adjacent to and immediately following the amino acid occupying a position. Non-limiting examples for variants of transcriptional regulator polypeptides are polypeptides comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO:24.

Viscosity: The term “viscosity” or “culture broth viscosity” means the dynamic or absolute viscosity of the culture broth being formed by cultivation of the host cells in cultivation medium. Viscosity is a measure of the resistance of a fluid to deformation by mechanical stress, such as shear stress or tensile stress. In the present context, viscosity can also refer to the resistance of a cell broth comprising filamentous fungus cells to mechanical stress, e.g., as provided by a rotor/impeller. Because the viscosity of a cell broth can be difficult to measure directly, indirect measurements of viscosity can be used, such as the dissolved oxygen content of the culture broth at a preselected amount of agitation, the amount of agitation required to maintain a preselected dissolved oxygen content, the amount of power required to agitate a cell broth to maintain a preselected dissolved oxygen content, or even colony morphology on solid medium.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### DETAILED DESCRIPTION OF THE INVENTION

### Host Cells

The present invention is also related to recombinant host cells, comprising a polynucleotide of the present invention operably linked to one or more heterologous control sequences that direct the expression of a fungal transcriptional regulator polypeptide or variant thereof. A construct or vector comprising a polynucleotide is introduced into a host cell so that the construct or vector is maintained as a chromosomal integrant or as a self-replicating extrachromosomal vector as described earlier. The choice of the host cell will to a large extent depend upon the gene encoding the polypeptide and its source.

In some embodiments, the transcriptional regulator polypeptide or variant thereof is heterologous to the recombinant host cell.

In preferred embodiments, the transcriptional regulator polypeptide or variant thereof is endogenous to the recombinant host cell.

In one embodiment, the transcriptional regulator polypeptide, or variant thereof, comprises at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:79 and/or SEQ ID NO:80.

In one embodiment, the transcriptional regulator polypeptide, or variant thereof, comprises at least one DNA-binding motif comprising or consisting of an amino acid sequence corresponding to amino acids 257-281 of SEQ ID NO:24.

In one embodiment, the transcriptional regulator polypeptide, or variant thereof, comprises at least one DNA-binding motif comprising or consisting of an amino acid sequence corresponding to amino acids 286-311 of SEQ ID NO:24.

In one embodiment, the transcriptional regulator polypeptide, or variant thereof, comprises at least one DNA-binding motif comprising or consisting of an amino acid sequence corresponding to amino acids 257-281 of SEQ ID NO:24 and at least one DNA-binding motif comprising or consisting of an amino acid sequence corresponding to amino acids 286-311 of SEQ ID NO:24.

In one embodiment, the transcriptional regulator polypeptide, or variant thereof, comprises at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:79; and at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:80.

In one embodiment the at least one DNA-binding motif comprises of, consists essentially of, or consists of the polypeptide with the amino acid of SEQ ID NO:79 and the polypeptide of SEQ ID NO:80.

In one embodiment the at least one DNA-binding motif comprises of, consists essentially of, or consists of the polypeptide with the amino acid of SEQ ID NO:79 and the polypeptide of SEQ ID NO:80, wherein one or both of said polypeptide sequences comprise at least one amino acid substitution, amino acid deletion and/or amino acid insertion. Preferably, the at least one amino acid substation is a conservative amino acid substitution.

In one embodiment, the at least one DNA-binding motif comprises an amino acid sequence selected from the list of “FzRyEHLKRH”, wherein y=N or Q, and z=R or K (amino acids corresponding to amino acids 268-277 of SEQ ID NO:24); and “RyDNLNxH” wherein x=N or A; and y=Q or S (amino acids corresponding to amino acids 300-307 of SEQ ID NO:24). Alternatively, the at least one DNA-binding motif comprises an amino acid sequence selected from the list of “FzRyEHLKRH”, wherein y=N or Q, and z=R or K (amino acids corresponding to amino acids 268-277 of SEQ ID NO:24); and “RyDNLNxH” wherein x=N or A; and y=Q or S (amino acids corresponding to amino acids 300-307 of SEQ ID NO:24), wherein the amino acid sequence from said list comprises one or more amino acid substitutions, preferably one or more conservative amino acid substitution.

In a preferred embodiment, the at least one DNA-binding motif comprises the amino acid sequences “FzRyEHLKRH”, wherein y=N or Q, and z=R or K (amino acids corresponding to amino acids 268-277 of SEQ ID NO:24); and “RyDNLNxH” wherein x=N or A; and y=Q or S (amino acids corresponding to amino acids 300-307 of SEQ ID NO:24).

In some embodiments, the recombinant host cell comprises at least two copies, e.g., three, four, or five copies of the polynucleotide encoding the transcriptional regulator polypeptide.

The host cell may be any microbial cell useful in the recombinant production of a polypeptide of interest, e.g. a fungal host cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

In a first aspect, the invention relates to fungal host cell comprising in its genome at least one first heterologous promoter operably linked to a first polynucleotide encoding a fungal transcriptional regulator polypeptide, or variant thereof, comprising or consisting of an amino acid sequence having a sequence identity of at least 60% to SEQ ID NO:24.

As presented throughout the examples, host cells comprising said at least one first heterologous promoter operably linked to a first polynucleotide have surprisingly shown increased expression of total host cell protein, increased expression of recombinant protein, and increased OUR associated with decreased culture broth viscosities.

In one embodiment, the at least one first heterologous promoter is heterologous to the first polynucleotide encoding the fungal transcriptional regulator polypeptide, or variant thereof.

In one embodiment of the first aspect, the transcriptional regulator polypeptide, or variant thereof, is endogenous to the host cell.

In one embodiment, the transcriptional regulator polypeptide or variant thereof is a regulator of xylanase regulator 1 (xyr1) expression, and/or a regulator of cellobiohydrolase 1 (cbh1) gene expression, preferably a regulator of the xyr1 promoter and/or a regulator of the cbh1 promoter.

In one embodiment, the transcriptional regulator polypeptide or variant thereof is a regulator of the xyr1 promoter and/or of the cbh1 promoter of a Trichoderma host cell.

In another embodiment, the transcriptional regulator polypeptide or variant thereof is a regulator of the xyr1 promoter and/or of the cbh1 promoter of a Trichoderma reesei host cell.

In a preferred embodiment of the first aspect, the transcriptional regulator polypeptide, or variant thereof, comprises or consists of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO:24.

In another embodiment, the transcriptional regulator polypeptide or variant thereof comprises, consists essentially of, or consists of SEQ ID NO: 24.

In one embodiment, the at least one first heterologous promoter operably linked to a first polynucleotide confers an increased level of the transcriptional regulator polypeptide, or variant thereof, to the host cell relative to an isogenic cell lacking said nucleic acid construct or expression vector.

In a further embodiment, the fungal host cell comprises in its genome at least one second heterologous promoter operably linked to at least one second polynucleotide encoding at least one polypeptide of interest. Preferably the at least one polypeptide of interest is secreted.

In one embodiment, the at least one first heterologous promoter and/or the at least one second heterologous promoter is a synthetic promoter.

In one embodiment, the productivity of the mutant in the production of the polypeptide of interest is increased at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% compared to an isogenic fungal cell not comprising the at least one first heterologous promoter operably linked to the first polynucleotide.

In one embodiment, the polynucleotide sequence of the second heterologous promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:42.

In one embodiment, the polynucleotide sequence of the second heterologous promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:45.

In one embodiment, the polynucleotide sequence of the second heterologous promoter comprises or consists of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:55.

In one embodiment, the fungal host cell comprises in its genome at least two first polynucleotides encoding the transcriptional regulator polypeptide, or variant thereof, such as two first polynucleotides, three first polynucleotides, four first polynucleotides, or more than four first polynucleotides encoding the transcriptional regulator polypeptide, or variant thereof. The number of first polynucleotides can be adjusted depending on the cultivation format, desired expression levels, type(s) of the at least one protein of interest, and the selected host cell.

In another embodiment, the first heterologous promoter operably linked to the first polynucleotide of the nucleic acid construct or expression vector is endogenous to the host cell.

In one embodiment, the first heterologous promoter comprises or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO:3.

In another embodiment, the first heterologous promoter is a constitutive promoter. Alternatively, the first heterologous promoter is a semi-constitutive promoter, or an inducible promoter.

In one embodiment, the first heterologous promoter comprises, consists essentially of, or consists of SEQ ID NO: 3.

In yet another embodiment, the first heterologous promoter is not native to the host cell.

In a preferred embodiment, the fungal host cell is a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus oryzae, Fusarium venenatum, and Trichoderma reesei cell; most preferably the filamentous fungal host cell is an Trichoderma reesei cell.

In a preferred embodiment, the host cell is a Trichoderma host cell, more preferably a Trichoderma reesei host cell.

In yet another embodiment, the host cell is a yeast host cell; preferably the yeast host cell is selected from the group consisting of Candida, Hansenula, Kluyveromyces, Pichia (Komagataella), Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell, most preferably the yeast host cell is Pichia pastoris (Komagataella phaffii).

In one embodiment, the at least one protein of interest is an endogenous protein of the host cell. Additionally or alternatively, the at least one protein of interest is at least two, at least three or at least four endogenous proteins of the host cell. Additionally or alternatively, the at least one protein of interest is the sum of all host cell proteins, preferably all secreted host cell proteins. In one embodiment, the at least one polypeptide of interest has no cellulase activity (EC 3.2.1.4).

In one embodiment, the at least one polypeptide of interest comprises a heme-containing polypeptide selected from the group consisting of a NADPH-cytochrome P450 oxidoreductase (EC 1.6.2.4); a cytochrome B (EC 1.10.2.2); a peroxidase (EC 1.11.1) such as a catalase (EC 1.11.1.6), a cytochrome-C peroxidase (EC 1.11.1.5) or peroxidases categorized as EC 1.11.1.7; a peroxygenase (EC 1.11.2), such as a haloperoxidase (EC 1.11.2.1); a plant peroxidase or a halo-peroxidase; a cytochrome P450 enzyme (EC 1.14.14.1), such as a P450 mono-oxygenase or a P450 di-oxygenase; a heme 35 oxygenase (EC 1.14.99.3); a ferredoxin reductase (EC 1.18.1.3); a cytochrome bd-I oxidase (Cytochrome-D; EC 7.1.1.7); and a cytochrome c-oxidase (cytochrome A; EC 7.1.1.9; former EC 1.9.3.1).

In one embodiment, the at least one polypeptide of interest comprises an active or an inactivated heme-containing enzyme selected from a list of polypeptides with at least 80% sequence identity to the polypeptides with SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, and SEQ ID NO:97.

In one embodiment, the at least one polypeptide of interest comprises a brazzein, a casein, a patatin, an ovalbumin, an osteopontin, an ovotransferrin, an ovomucin, an ovomucoid, an ovostatin, a glycomacropeptide, a lactoferrin, an alpha-lactalbumin, a beta-lactalbumin and/or a collagen.

In another embodiment, the at least one polypeptide of interest comprises a therapeutic polypeptide selected from the group consisting of an antibody, an antibody fragment, an antibody-based drug, a Fc fusion protein, an anticoagulant, a blood factor, a bone morphogenetic protein, an engineered protein scaffold, an enzyme, a growth factor, a blood clotting factor, a hormone, an interferon (such as an interferon alpha-2b), an interleukin, a lactoferrin, an alpha-lactalbumin, a beta-lactalbumin, an ovomucoid, an ovostatin, a cytokine, an obestatin, a human galactosidase (such as an human alpha-galactosidase A), a vaccine, a protein vaccine, and a thrombolytic.

In one embodiment, the at least one polypeptide of interest is selected from the group consisting of hydrolase, isomerase, ligase, lyase, lysozyme, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.

In a preferred embodiment, the at least one polypeptide of interest is a glycosylase, preferably a glycosidase, more preferably an amylase, cellobiohydrolase or mannosidase.

In another embodiment, the at least one polypeptide of interest is a hydrolase, preferably a glycosylase, more preferably a glycosidase; most preferably an amyloglucosidase (EC 3.2.1.3), such as an amyloglucosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:76.

In one embodiment, the at least one polypeptide of interest is a hydrolase, preferably a glycosylase; more preferably a glycosidase; most preferably a beta-mannosidase (EC 3.2.1.25), such as a beta-mannosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:77.

In another embodiment, the at least one polypeptide of interest is a hydrolase; preferably a glycosylase; more preferably a glycosidase; more preferably a cellobiohydrolase I or a cellobiohydrolase II (EC 3.2.1.91), such as a cellobiohydrolase I comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:78.

In one embodiment, at least two polypeptides of interest are encoded by the fungal host cell, wherein the at least two polypeptides of interested are selected from the list consisting of a cellobiohydrolase I comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:78, a beta-mannosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:77, and an amyloglucosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:76.

In another embodiment, at least three polypeptides of interest are encoded by the fungal host cell, wherein the at least three polypeptides of interest comprise a cellobiohydrolase I comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:78, a beta-mannosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:77, and an amyloglucosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:76.

In another embodiment, the first polynucleotide encoding the fungal transcriptional regulator polypeptide or variant thereof comprises one or more mutations, preferably nucleotide substitutions, nucleotide deletions or nucleotide insertions. Said mutation(s) leading to a variant of the transcriptional regulator polypeptide of SEQ ID NO: 24, such as a variant comprising (i) one or more additional amino acids compared to SEQ ID NO: 24, (ii) at least one amino acid less compared to SEQ ID NO: 24, e.g. a total of 10 to 20 amino acids, (iii) or an amino acid substitution of at least one amino acid of SEQ ID NO: 24, such as a conservative substitution of one or more amino acids at a position corresponding to positions 257 to 281 of SEQ ID NO:24, and/or a conservative substitution of one or more amino acids at a position corresponding to positions 286-311 of SEQ ID NO:24.

In one embodiment, the at least one substitution is a conservative amino acid substitution In some embodiments, the present invention relates to fungal host cells comprising at least one first heterologous promoter operably linked to a first polynucleotide encoding a polypeptide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 24, which is acting as a transcriptional regulator. In one aspect, the polypeptides differ by up to 10 amino acids, e.g., 1, 2, 3, 4, 5, 6, 7, 8, 9, or 10, from the mature polypeptide of SEQ ID NO:24.

The polypeptide preferably comprises, consists essentially of, or consists of the amino acid sequence of SEQ ID NO:24; or is a fragment thereof having transcriptional regulator activity. In one aspect, the mature polypeptide is SEQ ID NO: 24.

In some embodiments, the present invention relates to a first polynucleotide encoding the transcriptional regulator, wherein the first polynucleotide hybridizes under medium stringency conditions, medium-high stringency conditions, high stringency conditions, or very high stringency conditions with the full-length complement of the mature polypeptide coding sequence of SEQ ID NO:25 or the cDNA thereof (Sambrook et al., 1989, Molecular Cloning, A Laboratory Manual, 2d edition, Cold Spring Harbor, New York), such as SEQ ID NO:26.

The polynucleotide of SEQ ID NO: 25, SEQ ID NO:26 or a subsequence thereof, as well as the mature polypeptide of SEQ ID NO:24, or a fragment thereof, may be used to design nucleic acid probes to identify and clone DNA encoding polypeptides having transcriptional regulator activity from strains of different genera or species according to methods well known in the art. Such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having transcriptional regulator activity. Genomic or other DNA from such other strains may be separated by agarose or polyacrylamide gel electrophoresis, or other separation techniques. DNA from the libraries or the separated DNA may be transferred to and immobilized on nitrocellulose or another suitable carrier material. In order to identify a clone or DNA that hybridizes with SEQ ID NO:25, or SEQ ID NO:26 or a subsequence thereof, the carrier material is used in a Southern blot.

For purposes of the present invention, hybridization indicates that the polynucleotides hybridize to a labeled nucleic acid probe corresponding to (i) SEQ ID NO:25, or SEQ ID NO:26; (ii) the mature polypeptide coding sequence of SEQ ID NO:25, or SEQ ID NO:26; (iii) the full-length complement thereof; or (iv) a subsequence thereof; under medium to very high stringency conditions. Molecules to which the nucleic acid probe hybridizes under these conditions can be detected using, for example, X-ray film or any other detection means known in the art.

In some embodiments, the present invention relates to fungal host cells comprising at least one first heterologous promoter operably linked to a first polynucleotide encoding a fungal transcriptional regulator polypeptide, the first polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO:25, or SEQ ID NO:26.

The first polynucleotide encoding the polypeptide preferably comprises, consists essentially of, or consists of nucleotides 1 to 1160 of SEQ ID NO:25, or nucleotides 1 to 1092 of SEQ ID NO:26.

In some embodiments, the present invention relates to fungal host cells comprising at least one first heterologous promoter operably linked to a first polynucleotide encoding a fungal transcriptional regulator polypeptide derived from a mature polypeptide of SEQ ID NO:24 by substitution, deletion or addition of one or several amino acids in the mature polypeptide of SEQ ID NO:24. In some embodiments, the present invention relates to host cells comprising variants of the mature polypeptide of SEQ ID NO:24 comprising a substitution, deletion, and/or insertion at one or more (e.g., several) positions. In one aspect, the number of amino acid substitutions, deletions and/or insertions introduced into the mature polypeptide of SEQ ID NO: 24 is up to 10, e.g., 1, 2, 3, 4, 5, 6, 7, 8, 9, or 10. In one embodiment, the substitution is a conservative amino acid substitution. In an embodiment, the polypeptide has an N-terminal extension and/or C-terminal extension of 1-10 amino acids, e.g., 1, 2, 3, 4, 5, 6, 7, 8, 9, or 10 amino acids. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for transcriptional regulator activity to identify amino acid residues that are critical to the activity and/or DNA-binding specificity of the molecule. See also, Hilton et al., 1996, J. Biol. Chem. 271: 4699-4708. The mode of action of the regulator polypeptide or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related transcriptional regulator polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode transcriptional regulator polypeptides can be recovered from the host cells and rapidly sequenced using standard methods in the art. These methods allow the rapid determination of the importance of individual amino acid residues in a transcriptional regulator polypeptide.

In some embodiments, the transcriptional regulator polypeptide is a fragment containing at least 100 amino acid residues of the mature polypeptide of SEQ ID NO:24, at least 200 amino acid residues of the mature polypeptide of SEQ ID NO:24, at least 300 amino acid residues of the mature polypeptide of SEQ ID NO:24, or at least 350 amino acid residues of the mature polypeptide of SEQ ID NO:24.

In some embodiments, the transcriptional regulator polypeptide is a fragment containing at least one DNA-binding motif comprising or consisting of an amino acid sequence corresponding to amino acids 257-281 of SEQ ID NO:24 and at least one DNA-binding motif comprising or consisting of an amino acid sequence corresponding to amino acids 286-311 of SEQ ID NO:24.

### Methods of Production

In a second aspect, the present invention relates to a method for producing at least one polypeptide of interest, the method comprising:
(i) providing a fungal host cell according to the first aspect,
(ii) cultivating said fungal host cell under conditions conducive for expression of the at least one polypeptide of interest; and, optionally
(iii) recovering the at least one polypeptide of interest.

The host cells are cultivated in a nutrient medium suitable for production of the polypeptide using methods known in the art and as described in the Examples below. For example, the cells may be cultivated by shake flask (SF) cultivation, or small-scale or large-scale fermentation (including continuous, batch, fed-batch, or solid-state fermentations) in laboratory or industrial bioreactors in a suitable medium and under conditions allowing the at least one polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates. As shown throughout the examples, the inventors have surprisingly found that increased expression of the fungal transcriptional regulator polypeptide, results in increased activity, secretion and/or yield of the at least one polypeptide of interest.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide

The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Polynucleotides

The present invention also relates to isolated polynucleotides encoding a fungal transcriptional regulator polypeptide, or variant thereof, of the present invention, as described herein.

The techniques used to isolate or clone a polynucleotide are known in the art and include isolation from genomic DNA or cDNA, or a combination thereof. The cloning of the polynucleotides from genomic DNA can be affected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Aspergillus niger, Aspergillus oryzae, Trichoderma reesei, Rasamsonia emersonii, Pichia pastoris (Komagataella phaffii), or a related organism and thus, for example, may be a species variant of the polypeptide encoding region of the first polynucleotide.

Modification of a polynucleotide encoding a transcriptional regulator polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in DNA-binding affinity, DNA-binding specificity, RNA-polymerase recruitment, or the like. The variants may be constructed on the basis of the polynucleotide presented as the mature polypeptide coding sequence of SEQ ID NO:25 and SEQ ID NO:26, e.g. a subsequence thereof, and/or by introduction of nucleotide substitutions that do not result in a change in the amino acid sequence of the polypeptide, but which correspond to the codon usage of the host organism intended for production of the at least one polypeptide of interest, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

In a third aspect, the present invention relates to a nucleic acid construct comprising at least one first heterologous promoter operably linked to a first polynucleotide encoding a fungal transcriptional regulator polypeptide, or variant thereof.

In one embodiment of the third aspect, the transcriptional regulator polypeptide or variant thereof is a regulator of xylanase regulator 1 (xyr1) gene expression, and/or a regulator of cellobiohydrolase 1 (cbh1) gene expression, preferably a regulator of the xyr1 promoter and/or a regulator of the cbh1 promoter.

In one embodiment, the transcriptional regulator polypeptide, or variant thereof, comprises at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:79 and/or SEQ ID NO:80.

In one embodiment, the transcriptional regulator polypeptide, or variant thereof, comprises at least one DNA-binding motif comprising or consisting of an amino acid sequence corresponding to amino acids 257-281 of SEQ ID NO:24.

In one embodiment, the transcriptional regulator polypeptide, or variant thereof, comprises at least one DNA-binding motif comprising or consisting of an amino acid sequence corresponding to amino acids 286-311 of SEQ ID NO:24.

In one embodiment, the transcriptional regulator polypeptide, or variant thereof, comprises at least one DNA-binding motif comprising or consisting of an amino acid sequence corresponding to amino acids 257-281 of SEQ ID NO:24 and at least one DNA-binding motif comprising or consisting of an amino acid sequence corresponding to amino acids 286-311 of SEQ ID NO:24.

In one embodiment, the transcriptional regulator polypeptide, or variant thereof, comprises at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:79; and at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:80.

In one embodiment the at least one DNA-binding motif comprises of, consists essentially of, or consists of the polypeptide with the amino acid of SEQ ID NO:79 and the polypeptide of SEQ ID NO:80.

In one embodiment the at least one DNA-binding motif comprises of, consists essentially of, or consists of the polypeptide with the amino acid of SEQ ID NO:79 and the polypeptide of SEQ ID NO:80, wherein one or both of said polypeptide sequences comprise at least one amino acid substitution, amino acid deletion and/or amino acid insertion. Preferably, the at least one amino acid substation is a conservative amino acid substitution.

In one embodiment, the at least one DNA-binding motif comprises an amino acid sequence selected from the list of “FzRyEHLKRH”, wherein y=N or Q, and z=R or K (amino acids corresponding to amino acids 268-277 of SEQ ID NO:24); and “RyDNLNxH” wherein x=N or A; and y=Q or S (amino acids corresponding to amino acids 300-307 of SEQ ID NO:24).

In a preferred embodiment, the transcriptional regulator polypeptide, or variant thereof, comprises or consists of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO:24.

In one embodiment, the transcriptional regulator polypeptide or variant thereof comprises, consists essentially of, or consists of SEQ ID NO: 24.

In another embodiment, the first heterologous promoter is a constitutive promoter, a semi-constitutive promoter, or an inducible promoter.

In another embodiment, the first heterologous promoter operably linked to the first polynucleotide of the nucleic acid construct or expression vector is endogenous to the host cell.

In one embodiment the first heterologous promoter is heterologous to the first polynucleotide.

In one embodiment, the first heterologous promoter comprises or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO:3.

The polynucleotide may be manipulated in a variety of ways to provide for expression of the fungal transcriptional regulator polypeptide, or variant thereof. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous (native) or heterologous (non-native) to the host cell.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

In one embodiment, the at least one first heterologous promoter is a synthetic promoter.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, such as the terminators comprising or consisting of SEQ ID NO:44, SEQ ID NO:47, or SEQ ID NO:57.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′ end of the coding sequence of the polynucleotide may inherently contain a signal peptide coding sequence naturally linked in translation reading frame with the segment of the coding sequence that encodes the polypeptide.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in filamentous fungi systems include the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter. In yeast, the ADH2 system or GAL1 system may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the transcriptional regulator polypeptide, or variant thereof at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

In a fourth aspect, the present invention relates to an expression vector comprising a nucleic acid construct according to the third aspect.

The recombinant expression vector may be any vector (e.g., a plasmid or virus) that can be conveniently subjected to recombinant DNA procedures and can bring about expression of the polynucleotide encoding the transcriptional regulator polypeptide, or variant thereof. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the transcriptional regulator polypeptide, or variant thereof, or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase expression of the transcriptional regulator polypeptide, or variant thereof. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Methods for Generating a Recombinant Fungal Host Cell

In a fifth aspect, the present invention relates to methods for generating a recombinant fungal host cell with increased protein secretion relative to an isogenic cell, the method comprising:
i) providing a fungal host cell secreting at least one protein,
ii) providing the at least one nucleic acid construct according to the third aspect or the at least one expression vector according to the fourth aspect, and
iii) integrating the at least one nucleic acid construct or the at least one expression vector into the genome of the host cell, wherein the at least one nucleic acid construct or the at least one expression vector confers an increased level of the transcriptional regulator polypeptide, or variant thereof, to the recombinant host cell relative to an isogenic cell lacking said nucleic acid construct or expression vector.

Additionally or alternatively to steps ii) and/or iii), the transcriptional regulator polypeptide is native to the host cell, wherein expression of the native regulator polypeptide is increased, such as by using a method selected from the list of CRISPR activation, DNA methylation, RNA interference, a promoter-switch system, a promoter/transcription factor system, and transcriptional regulator copy-number increase.

In one embodiment, the productivity of the mutant in the production of the at least one secreted polypeptide is increased at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% compared to an isogenic fungal cell not comprising the at least one nucleic acid construct according to the third aspect or the at least one expression vector according to the fourth aspect.

In a preferred embodiment, the fungal host cell is a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus oryzae, Fusarium venenatum, and Trichoderma reesei cell; most preferably the filamentous fungal host cell is an Trichoderma reesei cell.

In a preferred embodiment, the host cell is a Trichoderma host cell, more preferably a Trichoderma reesei host cell.

In yet another embodiment, the host cell is a yeast host cell; preferably the yeast host cell is selected from the group consisting of Candida, Hansenula, Kluyveromyces, Pichia (Komagataella), Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell, most preferably the yeast host cell is Pichia pastoris (Komagataella phaffii).

In one embodiment, the method generates a host cell according to the first aspect of the invention.

In another embodiment, the method comprises an additional step iv) integrating into the genome of the host cell at least one heterologous polynucleotide encoding a polypeptide of interest. Additionally or alternatively, the polypeptide of interest is expressed in the host cells according to the first aspect.

In one embodiment, the invention is directed to a method for constructing a mutant of a parent fungal cell, comprising increasing the expression of one or more genes each encoding a transcriptional regulator polypeptide in the parent fungal cell to produce the mutant, wherein the parent fungal cell or the mutant thereof comprises a coding sequence of a polypeptide of interest under the transcriptional control of a promoter regulated by one or more of the transcriptional regulator polypeptides, wherein the one or more transcriptional regulator polypeptides are selected from the group consisting of:
(a) a transcriptional regulator comprising an amino acid sequence having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24;
(b) a transcriptional regulator encoded by a polynucleotide comprising a nucleotide sequence having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:25 or 26; and
(c) a transcriptional regulator encoded by a polynucleotide that hybridizes under high or very stringency conditions with the full-length complement of SEQ ID NO:25 or 26;
wherein the one or more transcriptional regulator genes are modified in the parent fungal cell to produce the mutant with increased production of the one or more transcriptional regulator, wherein (i) the modification of the expression of the one or more transcriptional regulator genes increases the productivity of the mutant in the production of the polypeptide of interest when cultivated under the same conditions as the parent fungal cell without the modification of the one or more transcriptional regulator genes, (ii) the modification of the one or more transcriptional regulator genes results in the formation of a culture broth with an increased oxygen uptake rate and/or a reduced viscosity, relative to the oxygen uptake rate and/or viscosity of a culture broth generated by the cultivation of the parental cell without the modification of the one or more transcriptional regulators, when cultivated under identical conditions, or (iii) the modification of the one or more transcriptional regulators genes results in a combination of (i) and (ii); and optionally recovering the mutant.

In one embodiment, the at least one nucleic acid construct or the at least one expression vector confers at least an 2-fold increase of the transcriptional regulator polypeptide, or variant thereof, to the recombinant host cell relative to an isogenic cell lacking said nucleic acid construct or expression vector.

### Methods for Aerobic Cultivation of Fungal Cells

In a sixth aspect, the present invention relates to methods for aerobic cultivation of recombinant fungal host cells, the method comprising:
i) providing a fungal host cell according to the first aspect,
ii) cultivating the mutated fungal host cell under aerobic conditions conducive for expression of the at least one polypeptide of interest,
   wherein the aerobic cultivation of the fungal host cells is characterized by the formation of a culture broth with an increased oxygen uptake rate and/or a reduced viscosity, relative to the oxygen uptake rate and/or viscosity of a culture broth generated by the cultivation of an isogenic fungal host cells lacking the at least one nucleic acid construct and/or the at least one expression vector, when cultivated under identical or similar conditions.

In one embodiment, the increased oxygen uptake rate is determined by measuring the dissolved oxygen in a cultivation system, preferably a bioreactor.

In one embodiment the oxygen uptake rate is increased by at least 10%, such as at least 15%, at least 20%, at least 25%, at least 30%, at least 35%, at least 40%, at least 45%, or at least 50% relative to the OUR of an isogenic cell when cultivated under identical or similar conditions.

In one embodiment, the increased oxygen uptake rate is determined by measuring the oxygen feed supplemented into the cultivation system or bioreactor during a predetermined duration.

In one embodiment the reduced viscosity is determined by measuring the total feed supplemented into the cultivation system or bioreactor during a predetermined duration.

In one embodiment the total feed supplemented to the aerobic cultivation process of the fungal host cell is increased by at least 10%, such as at least 15%, at least 20%, at least 25%, at least 30%, at least 35%, at least 40%, at least 45%, or at least 50% relative to the total feed supplemented to the cultivation process of an isogenic cell when cultivated under identical or similar conditions.

In one embodiment, the reduced viscosity and/or increased oxygen uptake rate is determined by a reduced amount of agitation to maintain a preselected dissolved oxygen content compared to isogenic fungal host cells.

Additionally or alternatively, the reduced viscosity and/or increased oxygen uptake rate is determined by maintenance of an increased dissolved oxygen content at a preselected amount of agitation, compared to the isogenic fungal host cells.

### Methods for Producing Transcriptional Regulator Polypeptides

In a seventh aspect, the present invention relates to methods for producing at least one transcriptional regulator polypeptide, the method comprising:
i) providing a fungal host cell according to the first aspect,
ii) cultivating said fungal host cell under conditions conducive for expression of the at least one transcriptional regulator; and
iii) optionally, recovering the at least one transcriptional regulator.

The host cells are cultivated in a nutrient medium suitable for production of the transcriptional regulator using methods known in the art and as described in the Examples below. For example, the cells may be cultivated by shake flask (SF) cultivation, or small-scale or large-scale fermentation (including continuous, batch, fed-batch, or solid-state fermentations) in laboratory or industrial bioreactors in a suitable medium and under conditions allowing the at least one polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the transcriptional regulator is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Uses of Transcriptional Regulators for Transcription Regulation

In an eight aspect, the invention relates to uses of a transcriptional regulator polypeptide for in-vitro transcription regulation, wherein the transcriptional regulator polypeptide is expressed by a fungal cell according to the first aspect, or wherein the transcriptional regulator polypeptide is produced by the method according to the seventh aspect.

In one embodiment the transcriptional regulator polypeptide comprises at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:79 and/or SEQ ID NO:80.

In one embodiment, the transcriptional regulator polypeptide, or variant thereof, comprises at least one DNA-binding motif comprising or consisting of an amino acid sequence corresponding to amino acids 257-281 of SEQ ID NO:24.

In one embodiment, the transcriptional regulator polypeptide, or variant thereof, comprises at least one DNA-binding motif comprising or consisting of an amino acid sequence corresponding to amino acids 286-311 of SEQ ID NO:24.

In one embodiment, wherein the transcriptional regulator polypeptide, or variant thereof, comprises at least one DNA-binding motif comprising or consisting of an amino acid sequence corresponding to amino acids 257-281 of SEQ ID NO:24 and at least one DNA-binding motif comprising or consisting of an amino acid sequence corresponding to amino acids 286-311 of SEQ ID NO:24.

In one embodiment, the transcriptional regulator polypeptide, or variant thereof, comprises at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:79; and at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:80.

In one embodiment, the at least one DNA-binding motif comprises of, consists essentially of, or consists of the polypeptide with the amino acid of SEQ ID NO:79 and the polypeptide of SEQ ID NO:80.

In one embodiment, the at least one DNA-binding motif comprises of, consists essentially of, or consists of the polypeptide with the amino acid of SEQ ID NO:79 and the polypeptide of SEQ ID NO:80, wherein one or both of said polypeptide sequences comprise at least one amino acid substitution, amino acid deletion and/or amino acid insertion. Preferably, the at least one amino acid substation is a conservative amino acid substitution.

In one embodiment, the at least one DNA-binding motif comprises an amino acid sequence selected from the list of “FzRyEHLKRH”, wherein y=N or Q, and z=R or K (amino acids corresponding to amino acids 268-277 of SEQ ID NO:24); and “RyDNLNxH” wherein x=N or A; and y=Q or S (amino acids corresponding to amino acids 300-307 of SEQ ID NO:24).

In one embodiment, the transcriptional regulator is used to regulate the transcription of a xyr1 promoter.

In one embodiment, the transcriptional regulator is used to regulate the transcription of a cbh1 promoter.

In one embodiment, the transcriptional regulator is used to regulate gene transcription in a cell-free expression system.

In one embodiment, the use of the transcriptional regulator results in increased in-vitro protein expression.

In one embodiment, the cell-free expression system comprises cellular components of a fungal host cell.

In one embodiment, the cell-free expression system comprises cellular components of a Trichoderma host cell.

In one embodiment, the cell-free expression system comprises cellular components of a Trichoderma reesei host cell.

In one embodiment, the cellular components of the cell-free expression system comprise one or more of a ribosome, a polymerase, at least one genomic DNA or DNA template, ATP, a cofactor, nucleotides, amino acids, and a tRNA.

### Methods for Producing Fungal Biomass

In a ninth aspect, the present invention relates to methods for producing a fungal biomass, the method comprising:
i) providing a fungal host cell according to the first aspect,
ii) cultivating said fungal host cell under conditions conducive for growth of the fungal host cell; and
iii) optionally, recovering the fungal host cell.

In one embodiment, the host cell does not express a heterologous polypeptide of interest.

In another embodiment, the host cell expresses a heterologous polypeptide of interest. In one embodiment, the heterologous polypeptide of interest is separated from the fungal host cell.

In a specific embodiment, the fungal biomass comprises or consists of the fungal host cell. The fungal biomass may be stored as wet, or as dry biomass.

The obtained fungal biomass may be utilized as a nitrogen source to enhance the subsequent fermentation with thermophilic bacteria for a high ethanol yield and productivity. Further uses of the fungal biomass include, but are not limited to, the extraction of biopolymers, with several applications in the food industry, cosmetics, and pharmaceutical, among others; and the removal of contaminants by mechanisms of adsorption with biopolymers, known also as biosorption, in tertiary treatments of wastewater.

Also, specific fungal biomass can have good nutritional value for supplementation to poultry, and be used in food or feed for different animal species. A further application of fungal biomass is its use in meat-free food and drink products.

### EXAMPLES

### Materials and Methods

Unless otherwise stated, DNA manipulations and transformations were performed using standard methods of molecular biology as described in Sambrook et al. (1989) Molecular cloning: A laboratory manual, Cold Spring Harbor lab., Cold Spring Harbor, NY; Ausubel, F. M. et al. (eds.) and “Current protocols in Molecular Biology”, John Wiley and Sons, 1995; Harwood, C. R., and Cut-ting, S. M. (eds.).

### Purchased Material

Amplified plasmids were recovered with Qiagen Plasmid Kit (Qiagen). DNA fragments were gel purified using the Qiagen MinElute Gel Extraction kit (Qiagen). Ligation reactions were carried out using the NEBUILDER® HiFi DNA Assembly Cloning Kit (New England Biolabs Inc.) according to the manufacturer's instructions. Polymerase Chain Reaction (PCR) was carried out with Phusion® DNA Polymerase (Thermo Fisher Scientific). Genomic DNA purification was carried out using the MAGMAX™ Plant DNA Kit (Thermo Scientific) and the KINGFISHER™ Duo Prime Machine (Thermo Scientific). Genomic DNA concentration was measured using a Qubit Fluorometric Quantification apparatus (Thermo Scientific). Genomic sequencing was carried out using the NEXTSEQ™ 500 System (Illumina Inc.). Sequence analysis was performed with the CLC Genomics Workbench version 11.0.1 (QIAGEN Inc.). PCR from genomic DNA was carried out using Thermo Scientific Phire Plant Direct PCR Kit (Thermo Scientific).

### Enzymes

Enzymes for DNA manipulations (e.g. restriction endonucleases, ligases etc.) were obtained from New England Biolabs, Inc. and were used according to the manufacturer's instructions.

### Plasmids

### Description of Plasmids D269AR and D269AT and Methods for Modification of Strain SAMF128-2A11-1 in Order to Create Strain O154NN.

Strain 0154NN was constructed by co-transforming SAMF128-2A11-1 with four plasmids in order to modify four loci in the genome simultaneously.

Plasmid D269AR contains the following nucleotide sequenced used for genome modification: a 700 bp segment of 5′ flanking sequence upstream of the native Trichoderma reesei Xy12 coding sequence (SEQ ID NO:27), an 8 bp synthetic spacer sequence (SEQ ID NO:28), a 988 bp segment of the Trichoderma reesei CbhI promoter (SEQ ID NO:29), the Rasamsonia byssochlamydoides Cellobiohydrolase 1 (CBH I) variant Rc-899 coding sequence (1705 bp) (SEQ ID NO:30) encoding the CBH I with SEQ ID NO:78, a 238 bp segment of the Trichoderma reesei CbhI terminator (SEQ ID NO:31), a 6 bp synthetic spacer sequence (SEQ ID NO:32), and a 700 bp segment of the 3′ flanking sequence downstream of the native Trichoderma reesei Xy12 coding sequence (SEQ ID NO:33).

The 5′ and 3′ Xy12 upstream and downstream gene flanking sequences contained within this plasmid D269AR were used for homologous recombination mediated, double stranded break repair at the Xy12 locus. The double stranded break was initiated by co-transformation of the cell with two plasmids pGMER263-fcy2proto and pGMER263-fyc3proto (described below) capable of CRISPR/Mad7 based fcyA targeted double stranded break generation at two sites in the fcyA gene which is located within the Xy12 flanks in the SAMF128-2A11-1 host.

Plasmid D269AT contains the following nucleotide sequenced used for genome modification: a 700 bp segment of 5′ flanking sequence upstream of the native Trichoderma reesei Cbh2 coding sequence (SEQ ID NO:34), an 8 bp synthetic spacer sequence (SEQ ID NO:35), a 988 bp segment of the Trichoderma reesei CbhI promoter (SEQ ID NO:36), the Rasamsonia byssochlamydoides Cellobiohydrolase 1 variant Rc-899 coding sequence (1705 bp) (SEQ ID NO:37), a 238 bp segment of the Trichoderma reesei CbhI terminator (SEQ ID NO:38), a 6 bp synthetic spacer sequence (SEQ ID NO:39), and a 700 bp segment of the 3′ flanking sequence downstream of the native Trichoderma reesei Cbh2 coding sequence (SEQ ID NO:40).

The 5′ and 3′ Cbh2 upstream and downstream gene flanking sequences contained within this plasmid D269AT were used for homologous recombination mediated, double stranded break repair at the Cbh2 locus. The double stranded break was initiated by co-transformation of the cell with two plasmids pGMER263-fcy2proto and pGMER263-fyc3proto (described below) capable of CRISPR/Mad7 based fcyA targeted double stranded break generation at two sites in the fcyA gene which is located within the Cbh2 flanks in the SAMF128-2A11-1 host.

As above, the double stranded breaks generated at the CbhI and EgI loci were initiated by co-transformation of the cell with two plasmids pGMER263-fcy2proto and pGMER263-fyc3proto (described below) capable of CRISPR/Mad7 based fcyA targeted double stranded break generation at two sites in the fcyA gene which is located within the CbhI and EgI flanks in the SAMF128-2A11-1 host. The CbhI and EgI loci were repaired using homologous recombination between the FRT-F and FRT-F3 sites that are present at these two loci in the SAMF128-2A11-1 host.

Description of Plasmid pGMER263 Containing the CRISPR/Mad7 Backbone Sequences Along with the Hygromycin Selection Marker and the AMA Sequence for Autonomous Replication in T. reesei.

Plasmid pGMEr263 was used as a backbone vector for genome editing in Trichoderma reesei. Plasmid pGMER263 is a CRISPR/MAD7 expression plasmid used to clone protospacers into Bgl II digested pGMER263 using an NEBUILDER® HiFi DNA Assembly Cloning Kit (New England Biolabs Inc.). Plasmid pGMEr263 contains the E. coli pUC19 sequence (nucleotides 1-331 bp; 331 bp, SEQ ID NO:52), the autonomous maintenance in Aspergillus (AMA1) sequence (Gems et al., 1991, Gene 98: 61-67) (nucleotides 332-6056) for extrachromosomal replication of pGMEr263 in T. reesei (nucleotide 332-6056 bp; 5725 bp, SEQ ID NO:53), synthetic linker sequence (nucleotide 6057-6081 bp; 25 bp, SEQ ID NO:54), the Coprinus cinereus beta tubulin promoter (nucleotide 6082-6474 bp; 393 bp, SEQ ID NO:55), the hygromycin phosphotransferase (hpt) gene from pHT1 (Cummings et al., 1999, Curr. Genet. 36: 371) conferring resistance to hygromycin B (nucleotide 6475-7506 bp; 1032 bp, SEQ ID NO:56), the Coprinus cinereus beta tubulin terminator (nucleotide 7507-7929 bp; 423 bp, SEQ ID NO:57), synthetic linker sequence (nucleotide 7930-7948 bp; 19 bp, SEQ ID NO:58), the Magnaporthe oryzae U6-2 promoter (nucleotides 7949-8448 bp; 500 bp, SEQ ID NO:59), the Aspergillus fumigatus tRNAgly(GCC)1-6 sequence with the region downstream of the structural tRNA removed (nucleotides 9449-8539 bp; 91 bp, SEQ ID NO:60), the E. rectale single guide RNA sequence (nucleotides 8540-8560 bp; 21 bp, SEQ ID NO:61), the M. oryzae U6-2 terminator (nucleotides 8561-8776 bp; 216 bp, SEQ ID NO:62), the Aspergillus nidulans tef1 promoter (nucleotides 8777-9662 bp; 886 bp, SEQ ID NO:63) from pFC330-333 (Nødvig et al., 2015, PLoS One 10(7): 1-18), the Eubacterium rectale Mad7 protein coding sequence (nucleotides 9663-13478 bp; 3816 bp, SEQ ID NO:64) codon-optimized for use in Aspergillus niger and a SV40 nuclear localization signal (NLS; nucleotides 13455-13475; 21 bp, SEQ ID NO:65) at the 3′ end of the E. rectale Mad7 open reading frame to ensure that Mad7 is localized to the nucleus, the Aspergillus nidulans tef1 terminator (nucleotides 13479-13883 bp; 405 bp, SEQ ID NO:66) from pFC330-333 (Nødvig et al., 2015, PLoS One 10(7): 1-18), and the E. coli pUC19 Ori and Ampicillin resistance marker (nucleotides 13884-163548621 bp; 2471 bp, SEQ ID NO:67). The plasmid was confirmed using DNA sequencing with a Model 377 XL Automated DNA Sequencer (Applied Biosystems Inc.) using dye-terminated chemistry.

Plasmid pGMEr263 was used as a backbone vector for genome editing in T. reesei. Plasmid pGMEr263 is a MAD7 expression plasmid used to clone protospacers into Bgl II digested pGMEr263 using an NEBUILDER® HiFi DNA Assembly Cloning Kit (New England Biolabs Inc.). Plasmid pGMEr263 contains a Eubacterium rectale Mad7 protein coding sequence (nucleotides 9663-13,478, SEQ ID NO:64) codon-optimized for use in Aspergillus niger and a SV40 nuclear localization signal (NLS; nucleotides 13,455-13,478, SEQ ID NO:65) at the 3′ end of the E. rectale Mad7 open reading frame to ensure that Mad7 is localized to the nucleus. Expression of the E. rectale Mad7 is under control of the Aspergillus nidulans tef1 promoter (nucleotides 8777-9662, SEQ ID NO:63) and tef1 terminator (nucleotides 13,479-13,883 of SEQ ID NO:66) from pFC330-333 (Nødvig et al., 2015, PLoS One 10(7): 1-18).

Plasmid pGMEr263 also has all the elements for single guide RNA (sgRNA) expression, which consists of the Magnaporthe oryzae U6-2 promoter (nucleotides 7949-8448, SEQ ID NO:59), Aspergillus fumigatus tRNAgly(GCC)1-6 sequence with the region downstream of the structural tRNA removed (nucleotides 8449-8539, SEQ ID NO:60), E. rectale single guide RNA sequence (nucleotides 8540-8560, SEQ ID NO:61), Bgl II restriction enzyme recognition sequence (nucleotides 8557-8562), and M. oryzae terminator (nucleotides 8561-8776, SEQ ID NO:62).

For selection in T. reesei, plasmid pGMEr263 contains the hygromycin phosphotransferase (hpt) gene from pHT1 (Cummings et al., 1999, Curr. Genet. 36: 371) (nucleotides 6475-7506, SEQ ID NO:56), conferring resistance to hygromycin B, and the autonomous maintenance in Aspergillus (AMA1) sequence (Gems et al., 1991, Gene 98: 61-67) (nucleotides 332-6056, SEQ ID NO:35) for extrachromosomal replication of pGMEr263 in T. reesei. The hygromycin resistance gene is under transcriptional control of the Coprinus cinereus beta-tubulin promoter (nucleotides 6082-6474, SEQ ID NO:55) and terminator (nucleotides 7503-7929, SEQ ID NO:57). The single guide RNA and the Mad7-SV40 NLS expression elements in pGMEr263 were confirmed by DNA sequencing with a Model 377 XL Automated DNA Sequencer (Applied Biosystems Inc.) using dye-terminator chemistry (Giesecke et al., 1992, J. Virol. Methods 38(1): 47-60).

Description of Plasmids pGMER263-fcy2proto and pGMER263-fcy3proto Used for MAD7 Targeting to the fcyA Gene.

The plasmid pGMER263 was digested with Bgl II and gel purified using Qiagen MiniElute Gel Extracton kit. Plasmids pGMER263-fyc2proto and pGMER263-fcy3proto were created by combining 100 ng of Bgl II digested pGMER263, 1 ul of 10 uM oligo 1232807 with SEQ ID NO:68 for pGMER263-fcy2 or 1 ul of 10 uM oligo GMER263_fcy3 (SEQ ID NO:72) for pGMER263-fcy3 and vector homology sequences using the NEBUILDER® HiFi DNA Assembly Cloning Kit (New England Biolabs Inc.).

The fcy2 protospacer with SEQ ID NO:69 guides the endonuclease to a more central region of the FcyA gene which is located in the SAMF128-2A11-1 genome. The 5′ homology sequence to pGMER263 at the BglII site is disclosed as SEQ ID NO:70, whereas the 3′ homology sequence to pGMER263 at the BglII site is disclosed as SEQ ID NO:71.

The fcy3 protospacer with SEQ ID NO:73 guides the endonuclease to the 3′ end of the FcyA gene which is located at 4 regions in the SAMF128-2A11-1 genome. The 5′ homology sequence to pGMER263 at the BglII site is disclosed as SEQ ID NO:74. The 3′ homology sequence to pGMER263 at the BglII site is disclosed as SEQ ID NO:75.

The plasmids pGMER263-fyc2proto and pGMER263-fcy3proto were confirmed to contain the fcy2 or fcy3 protospacer sequence by DNA sequencing with a Model 377 XL Automated DNA Sequencer (Applied Biosystems Inc.) using dye-terminated chemistry.

Description of Sequence from Plasmid D27WET that was Integrated into Strain O154NN in Order to Create Strain O16VA2

Plasmid D27WET contains the following nucleotide sequenced used for genome modification: a 1522 bp segment of 5′ flanking sequence of the native Trichoderma reesei Cbh1 coding sequence (SEQ ID NO:41), a 1000 bp segment of the Trichoderma viride CbhI promoter (SEQ ID NO:42), the Penicillium oxalicum amyloglucosidase Coding sequence (described in WO2011/127802) (1851 bp, SEQ ID NO:43) encoding the amyloglucosidase with SEQ ID NO:76, a 300 bp segment of the Trichoderma viride CbhI terminator (SEQ ID NO:44), a 1000 bp segment of the Trichoderma harzianum CbhI promoter (SEQ ID NO:45), the Aspergillus niger beta-mannosidase Coding sequence (3021 bp, SEQ ID NO:46) encoding the mannosidase with SEQ ID NO:77, a 300 bp segment of the Trichoderma harzianum CbhI terminator (SEQ ID NO:47), the hygromycin selection marker gene, promoter and terminator (1852 bp, SEQ ID NO:48), and a 1557 bp segment of the 3′ flanking sequence of the native Trichoderma reesei CbhI coding sequence (SEQ ID NO:49). The 5′ and 3′ CbhI gene flanking sequences contained within this plasmid were used for homologous recombination and subsequent integration of the intervening plasmid sequence.

Description of Plasmid D278ZE Containing the Native Trichoderma reesei gpdA Promoter and CbhI Terminator Containing Homology Flanks to the 70883 Locus for Integration and Disruption of this Locus.

Plasmid D278ZE was used as a backbone vector for genome editing in Trichoderma reesei. Plasmid D278ZE contains the following: E. coli pUC19 backbone sequence (nucleotides 1-454 bp; 454 bp, SEQ ID NO:1), Trichoderma reesei 5′ 70883 locus flanking sequence (nucleotides 455-2514 bp; 2060 bp, SEQ ID NO:2), Trichoderma reesei gpdA promoter sequence (nucleotides 2515-3496 bp; 982 bp, SEQ ID NO:3) (Martinez D. et al., Nat Biotechnol. 2008 May; 26(5):553-60. doi: 10.1038/nbt1403), synthetic linker DNA containing the restriction enzyme recognition sequences for NotI and Pac I (nucleotides 3497-3533 bp; 37 bp, SEQ ID NO:4), Trichoderma reesei CbhI terminator sequence (nucleotides 3534-3772 bp; 239 bp, SEQ ID NO:5) (Martinez D. et al., Nat Biotechnol. 2008 May; 26(5):553-60. doi: 10.1038/nbt1403), Aspergillus nidulans AmdS gene, promoter and terminator sequence (nucleotides 3773-6490 bp; 2718 bp, SEQ ID NO:6), Trichoderma reesei 3′ 70883 locus flanking sequence (nucleotides 6491-8526 bp; 2036 bp, SEQ ID NO:7), and E. coli pUC19 backbone and ampicillin resistance selection marker sequence (nucleotides 8527-10,768 bp; 2242 bp, SEQ ID NO:8).

Construction of Plasmid D27XZX for Integration and Over-Expression of the Native Protein 108357 with SEQ ID NO:24 in Trichoderma reesei Using the Native Trichoderma reesei gpdA Promoter and CbhI Terminator at the 70883 Locus while Also Disrupting the 70883 Locus.

The plasmid D27XZX derived from plasmid D278ZE, contains the nucleotide sequence with SEQ ID NO:11 encoding the Trichoderma reesei transcriptional regulator polypeptide with SEQ ID NO:24 corresponding to JGI protein ID 108357 (Martinez D. et al., Nat Biotechnol. 2008 May; 26(5):553-60. doi: 10.1038/nbt1403) inserted between the Trichoderma reesei glyceraldehyde-3-phosphase-dehydrogenase/promoter and cellobiohydrolase/terminator, was constructed as follows. The approximately 1.2 kb region corresponding to the coding sequence corresponding to JGI protein ID 108357 was amplified from genomic DNA of BTR213, disclosed as SEQ ID NO:25 (BTR213 has been previously described in WO 2013086633) by PCR with the corresponding primer pairs (NZGP_EFP1DCDXMW_fwd disclosed as SEQ ID NO:9 and NZGP_EFP1DCDXMW_rev disclosed as SEQ ID NO:10), see Table 1. The cDNA of BTR213 is disclosed as SEQ ID NO:26.

The obtained 1.2 kb DNA fragment was ligated with the PacI/NotI digested plasmid D278ZE using the NEBuilder® HiFi DNA Assembly Master Mix (New England Biolabs) according to the manufacture's protocol, to create a single expression plasmid D27XZX. The plasmid was confirmed using DNA sequencing with a Model 377 XL Automated DNA Sequencer (Applied Biosystems Inc.) using dye-terminated chemistry.

TABLE 1
PCR amplifications

| | | |
|---|---|---|
| | | Final |
| Component | Volume | Concentration |

| 5x Phusion GC buffer | 5 | μl | 1x | |
|---|---|---|---|---|
| 10 mM dNTPs | 0.5 | μl | 0.2 mM each | |
| 5 uM Primer | 2.5 | μl | 0.5 | μM |
| NZGP_EFP1DCDXMW_fwd | | | | |
| 5 uM Primer | 2.5 | μl | 0.5 | μM |
| NZGP_EFP1DCDXMW_rev | | | | |
| Template DNA | 1 | μl | 4 | ng/ul |
| Genomic DNA (BTR213) | 100 | ng/μl | | |
| DMSO | 0.75 | ul | 3% | |
| Phusion Polymerase (2 U/ul) | 0.25 | μl | 0.5 | U |
| PCR grade water | 6.5 | μl | | |
| Total reaction volume | 25 | μl | | |

3-step Cycle:
Step 1: Pre-denaturation: 98° C., 30 sec
Step 2: Denaturation: 98° C., 10 sec.
Step 3: Annealing: 65° C., 10 sec.
Step 4: Extension: 72° C., 4 min.
Step 5: Repeat steps 2-4, 34 times
Step 6: Final Extension: 72° C., 10 min.
   Description of Plasmid pGMER259 Containing the CRISPR/Mad7 Backbone Sequences.

Plasmid pGMEr259 was used as a backbone vector for genome editing in Trichoderma reesei. Plasmid pGMER259 is a CRISPR/MAD7 expression plasmid used to clone protospacers into Bgl II digested pGMER259 using an NEBUILDER® HiFi DNA Assembly Cloning Kit (New England Biolabs Inc.). Plasmid pGMEr259 contains the E. coli pUC19 sequence (nucleotides 1-452 bp; 452 bp, SEQ ID NO: 12), the Magnaporthe oryzae U6-2 promoter (nucleotides 453-952 bp; 500 bp, SEQ ID NO:13), the Aspergillus fumigatus tRNAgly(GCC)1-6 sequence with the region downstream of the structural tRNA removed (nucleotides 953-1043 bp; 91 bp, SEQ ID NO:14), the E. rectale single guide RNA sequence (nucleotides 1044-1064 bp; 21 bp, SEQ ID NO:15), the Bgl II restriction enzyme recognition sequence (nucleotides 1061-1066 bp; 6 bp, SEQ ID NO:16), the M. oryzae U6-2 terminator (nucleotides 1066-1280 bp; 215 bp, SEQ ID NO:17), the Aspergillus nidulans tef1 promoter (nucleotides 1281-2166 bp; 886 bp, SEQ ID NO:18) from pFC330-333 (Nødvig et al., 2015, PLoS One 10(7): 1-18), the Eubacterium rectale Mad7 protein coding sequence (nucleotides 2167-5982 bp of SEQ ID NO:19; 3816 bp) codon-optimized for use in Aspergillus niger and a SV40 nuclear localization signal (NLS; nucleotides 5959-5979 of SEQ ID NO:19; 21 bp) at the 3′ end of the E. rectale Mad7 open reading frame to ensure that Mad7 is localized to the nucleus, the Aspergillus nidulans tef1 terminator (nucleotides 5983-6387 bp; 405 bp, SEQ ID NO:20) from pFC330-333 (Nødvig et al., 2015, PloS One 10(7): 1-18), and the E. coli pUC19 Ori and Ampicillin resistance marker (nucleotides 6388-8621 bp; 2234 bp, SEQ ID NO:21). The plasmid was confirmed using DNA sequencing with a Model 377 XL Automated DNA Sequencer (Applied Biosystems Inc.) using dye-terminated chemistry.

### Construction of Plasmid D26V2Q Containing the CRISPR/Mad7 Backbone Sequences as Well as Containing the Protospacer Sequence for Targeting Mad7 to the 70883 Locus

The plasmid pGMER259 was digested with Bgl II and gel purified using Qiagen MiniElute Gel Extracton kit. Plasmid D26V2Q was created by combining 100 ng of Bgl II digested pGMER259, 1 ul of 10 uM oligo 1231115 (SEQ ID NO:22) containing 70883 protospacer sequence (disclosed as SEQ ID NO:23) and vector homology sequences using the NEBUILDER® HiFi DNA Assembly Cloning Kit (New England Biolabs Inc.). The plasmid D26V2Q was confirmed to contain the 70883 protospacer sequence by DNA sequencing with a Model 377 XL Automated DNA Sequencer (Applied Biosystems Inc.) using dye-terminated chemistry.

### Media and Solutions

COVE plates were composed of 342.3 g of sucrose, 20 ml of COVE salts solution, 10 ml 1 M acetamide, 10 ml of 1.5 M CsCl, 25 g of Noble agar, and deionized water to 1 liter.

COVE2 plates were composed of 30 g of sucrose, 20 ml of COVE salts solution, 10 ml 1 M acetamide, 25 g of Noble agar, and deionized water to 1 liter.

COVE salts solution was composed of 26 g of KCl, 26 g of MgSO₄·7H₂O, 76 g of KH₂PO₄, 50 ml COVE trace metals solution, and deionized water to 1 liter.

COVE trace metals solution was composed of 0.04 g of Na₂B₄O·10H₂O, 0.4 g of CuSO₄·5H₂O, 1.2 g of FeSO₄·7H₂O, 0.7 g of MnSO₄·H₂O, 0.8 g of Na₂MoO₂·2H₂O, 10 g of ZnSO₄·7H₂O, and deionized water to 1 liter.

Fermentation batch medium was composed of 15.1 g of dextrose, 40 g of soy grits, 8 g of (NH₄)₂SO₄, 3 g of K₂HPO₄, 8 g of K₂SO₄, 3 g of CaCO₃, 8 g of MgSO₄·7H₂O, 1 g of citric acid·H₂O, 5.2 ml of 85% phosphoric acid, 1 ml of anti-foam, 14.7 ml of trace metals solution, and deionized water to 1 liter. The trace metals solution was composed of 26.1 g of FeSO₄·7H₂O, 5.5 g of ZnSO₄·7H₂O, 6.6 g of MnSO₄—H₂O, 2.6 g of CuSO₄·5H₂O, 2 g of citric acid·H₂O, and deionized water to 1 liter.

PDA plates were composed of 39 g of potato dextrose agar (Difco) and deionized water to 1 liter.

PEG+G buffer was composed of 60% polyethylene glycol (PEG) 4000, 20% w/v glucose, 10 mM Tris-HCl pH 7.5, and 10 mM CaCl₂ in deionized water. The solution is filter sterilized.

Shake flask medium was composed of 20 g of glycerol, 10 g of soy grits, 10 g of (NH₄)₂SO₄, 2 g of KH₂PO₄, 4 g of MgSO₄·7H₂O, 0.5 g CaCO₃, 0.2 ml of trace metals solution, and deionized water to 1 liter. The trace metals solution was composed of 26.1 g of FeSO₄·7H₂O, 5.5 g of ZnSO₄·7H₂O, 6.6 g of MnSO₄·H₂O, 2.6 g of CuSO₄·5H₂O, 2 g of citric acid·H₂O, and deionized water to 1 liter.

STC+G was composed of 1 M sorbitol, 20% w/v glucose, 10 mM Tris pH 7.5, and 10 mM CaCl₂ in deionized water.

STC was composed of 1 M sorbitol, 10 mM Tris pH 7.5, and 10 mM CaCl₂ in deionized water.

YPD medium was composed of 1% yeast extract, 2% peptone, and 2% glucose in deionized water.

### Automated Total Protein Assay.

This method was performed on a Thermo Scientific Gallery Analyzer (Thermo Scientific, Waltman MA). Cultures were diluted appropriately in water. Albumin standard (BSA) was serial diluted starting with to a concentration range of 0.66 mg/ml and ending with a 0.087 mg/ml in water. A total of 20 μl of each dilution including standard was transferred to a cuvette containing 200 μl of a bicinchoninic acid (BCA) substrate solution (Pierce BCA Protein Assay Kit: Thermo Scientific, Waltman MA) then incubated at 37° C. for 30 minutes. Upon completion of the incubation an optical density of 540 nm was obtained for each sample. Sample concentrations were determined by extrapolation from the generated standard curve.

Automated pN-AMG Assay.

Culture supernatants were diluted appropriately in 0.1M Na-acetate, 0.01% Triton X-100 buffer pH 5.0 (sample buffer) and placed in an empty 96-well plate. An assay standard was also diluted appropriately with sample buffer and was added to an empty column of the same plate with the samples. An additional 3-fold and 9-fold dilutions of the samples and standards were carried out and 20 μl of each dilution were placed into a new 96-well plate. Samples/standards were then incubated with 100 micro-liters of a p-Nitrophenyl-a-D-glucopyranoside substrate solution (1 mg/ml in 0.1M Na-acetate, pH 5.0) for period of 45 minutes at ambient temperature. Upon completion of the incubation the reaction was quenched with 100 μl of 0.06N NaOH prior to reading an optical density of 405 nm. The sample concentrations were extrapolated from the generated standard curve.

Automated β-mannosidase Assay.

Culture supernatants were diluted appropriately in 0.1M Sodium acetate, 4 mM CaCl2, 0.01% Triton X-100 buffer pH 6.0 (sample buffer) and placed in an empty 96-well plate. An assay standard is also diluted appropriately with sample buffer and is added to an empty column of the same plate with the samples. An additional 3-fold and 9-fold dilutions of the samples and standards were carried out and 20 μl of each dilution were placed into a new 96-well plate. Samples/standards were then incubated with 200 μl of a 1 mg/ml para-nitrophenyl-β-mannopyranoside (Sigma N1268) substrate in sample buffer for period of 45 minutes at ambient temperature. Upon completion of the incubation the reaction was quenched with 50 μl of 1M TRIS buffer pH9 prior to reading an optical density of 405 nm. The sample concentrations were extrapolated from the generated standard curve.

### Automated MUL Assay to Measure CBH I Activities.

Samples were diluted appropriately in 100 mM MOPS pH7 with 0.01% Triton X100 (assay buffer) and placed in an empty 96-well plate. An assay standard was also diluted appropriately with sample buffer and was added to an empty column of the same plate with the samples. An additional 3-fold and 9-fold dilutions of the samples and standards was carried out and 20 μl of each dilution was placed into a new 96-well plate. Samples/standards were then incubated with 200 μl of a 4-Methylumbelliferyl b-D-lactoside (MUL)—Stock @ 200 mg/ml in DMSO diluted 2,000-fold to 0.1 mg/ml in 100 mM Succinic acid pH5.0 for period of 11.5 minutes at ambient temperature. Upon completion of the incubation the reaction was quenched with 50 μl of 4% NaOH prior to a fluorescent read at EX368 nm/Em448 nm. The sample concentrations were extrapolated from the generated standard curve.

### Microbial Strains

Trichoderma reesei strain BTR213 is described in WO 2013086633.

Trichoderma reesei strain SAMF128-2A11-1 is described in WO 20112911.

### Example 1: Generation of Fungal Host Cells Expressing an Additional Copy of a Fungal Transcriptional Regulator

### Trichoderma Mutant Strains

Trichoderma reesei strain O154NN is derived from SAMF128-2A11-1 with the following modifications: (1) the FRT-F/FRT-F3 recognition sequence and intervening sequence have been deleted from the cellobiohydralase I and endoglucanase I loci and (2) the FRT-F/F3 recognition sequence and intervening sequence have been deleted from the cellobiohydrolase II and xylanase 2 loci and replaced with an expression cassette for heterologous expression of the Rasamsonia byssochlamydoides Cellobiohydrolase I variant Rc-899 coding sequence (previously described in WO2016037096A1). This strain was constructed using CRISPR based technology and the cells native homologous recombination machinery where the CRISPR ds break in the genome was repaired using the loci flanks present on the plasmid provided during transformation as described above using a CRISPR-based technology. The cellobiohydrolase I and endoglucase I loci were repaired through homology at the FRT sites. The cellobiohydrolase II and xylanase II loci were repaired using homology flanks presented on plasmids D269AT and D269AR.

Trichoderma reesei strain O253QJ is derived from strain O154NN where the 70883 coding sequence has been deleted and replaced with an expression cassette containing the Trichoderma reesei glyceraldehyde-3-phosphase-dehydrogenase I promoter, the Trichoderma reesei 108357 coding sequence, the Trichoderma reesei cellobiohydrolase I terminator, and containing the amdS selection marker. This strain was constructed using CRISPR based technology and the cells native homologous recombination machinery where the CRISPR ds break in the genome was repaired using the loci flanks present on the plasmid provided during transformation. The 70883 locus was repaired using homology flanks presented on plasmid D27XZX as described above using a CRISPR-based technology.

Trichoderma reesei strain O16E5W is derived from strain O154NN where the Trichoderma reesei 70883 coding sequence has been deleted and replaced with an empty expression cassette containing the Trichoderma reesei glyceraldehyde-3-phosphase-dehydrogenase I promoter, the Trichoderma reesei cellobiohydrolase/terminator, and containing the amdS selection marker. This strain was constructed using CRISPR based technology and the cells native homologous recombination machinery where the CRISPR ds break in the genome was repaired using the loci flanks present on the plasmid provided during transformation. The 70883 locus was repaired using homology flanks presented on plasmid D278ZE as described above using a CRISPR-based technology.

Trichoderma reesei strain O16VA2 is derived from strain O154NN and contains a multi-gene expression cassette for heterologous expression of the Penicillium oxalicum glucoamylase, the Aspergillus niger beta-mannosidase at the cellobiohydrolase I locus and contains the hygromycin B selection marker. This strain was constructed using CRISPR based technology and the cells native homologous recombination machinery where the CRISPR ds break in the genome was repaired using the loci flanks present on the plasmid provided during transformation. The cellobiohydrolase I locus was repaired using homology flanks presented on plasmid D27WET.

Trichoderma reesei strain O184PQ is derived from strain O16VA2 where the 70883 coding sequence has been deleted and replaced with an expression cassette containing the Trichoderma reesei glyceraldehyde-3-phosphase-dehydrogenase I promoter, the Trichoderma reesei 108357 coding sequence, the Trichoderma reesei cellobiohydrolase I terminator, and containing the amdS selection marker. This strain was constructed using CRISPR based technology and the cells native homologous recombination machinery where the CRISPR ds break in the genome was repaired using the loci flanks present on the plasmid provided during transformation. The 70883 locus was repaired using homology flanks presented on plasmid D27XZX as described above using a CRISPR-based technology.

Trichoderma reesei strain O1792Q is derived from strain O16VA2 where the Trichoderma reesei 70883 coding sequence has been deleted and replaced with an empty expression cassette containing the Trichoderma reesei glyceraldehyde-3-phosphase-dehydrogenase/promoter, the Trichoderma reesei cellobiohydrolase/terminator, and containing the amdS selection marker. This strain was constructed using CRISPR based technology and the cells native homologous recombination machinery where the CRISPR ds break in the genome was repaired using the loci flanks present on the plasmid provided during transformation. The 70883 locus was repaired using homology flanks presented on plasmid D278ZE as described above using a CRISPR-based technology.

Generation of Trichoderma reesei protoplasts

Protoplast Preparation and Transformation of Trichoderma reesei were Performed Using a Protocol similar to Penttila et al., 1987, Gene 61: 155-164. Briefly, T. reesei was cultivated in two shake flasks, each containing 25 ml of YPD medium, at 27° C. for 17 hours with gentle agitation at 90 rpm. Mycelia were collected by filtration using a Vacuum Driven Disposable Filtration System (Millipore) and washed twice with deionized water and twice with 1.2 M sorbitol. Protoplasts were generated by suspending the washed mycelia in 30 ml of 1.2 M sorbitol containing 5 mg of YATALASE™ (Takara Bio USA, Inc.) per ml and 0.5 mg of chitinase (Sigma Chemical Co.) per ml for 60-75 minutes at 34° C. with gentle shaking at 90 rpm. Protoplasts were collected by centrifugation at 834×g for 7 minutes and washed twice with cold 1.2 M sorbitol. The protoplasts were counted using a hemocytometer and re-suspended to a final concentration of 1×10⁸ protoplasts per ml of STC. Aliquots (1.1 ml) of the protoplast solution were placed in a MR. FROSTY™ freezing container (Thermo Fisher Scientific) at −80° C. for later use (as described in WO20123845).

### Trichoderma Transformation

Transformation of Trichoderma species can be achieved using the general methods for yeast transformation. The preferred procedure for this invention is described below. Approximately 1 μg of D27XZX or D278ZE plasmid DNA and 1 μg of plasmid D26V2Q DNA were combined (and added to 100 μl of the protoplast suspension of strain O16VA2 or 0154NN and then mixed gently. Then 250 μl PEG+G was added to the DNA-protoplast mixture, mixed gently and incubated at 34° C. for 30 minutes. Two mis of STC+G was added, the protoplast suspension was mixed gently and poured onto Cove agar plates. The plates were incubated at 30° C. for 8-10 days. Transformants were picked to Cove2 agar and incubated at 30° C. for 5-7 days. The strains were spore purified by diluting spores from the Cove2 agar plates in water and spreading onto Cove agar for a second round of selection (like described in WO20123845).

A portion of the D27XZX or D278ZE plasmid DNA was integrated into the genome by homologous recombination using the 5′ and 3′ flanks of the 70883 locus contained in the plasmids. The plasmid sequence between these 70883 homology flanks was integrated into the genome replacing the 70883 coding sequence. Transformants were selected for using the amdS selection marker contained between the 70883 flanking sequence within the plasmid. The resulting strains with the 70883 replacement and integrated intervening plasmid DNA sequence were named O184PQ (D27XZX in O16VA2), O1792Q (D278ZE in O16VA2), O253QJ (D27XZX in O154NN) and O16E5W (D278ZE in O154NN).

### Example 2: Whole Genome Sequencing of Mutant Trichoderma reesei Strains

Each of the mutant T. reesei strains O16E5W, O253QJ, O184PQ and O1792Q were grown in 5 ml of YPD medium in 14 ml tubes for 2 days at 30° C. with shaking at 300 rpm. The mycelia were collected by centrifugation and the genomic DNA was purified using a MAGMAX™ Plant DNA Kit (Thermo Scientific) in a KINGFISHER™ Duo Prime (Thermo Scientific). The final genomic DNA concentration was measured using a Qubit Fluorometric Quantification apparatus (Thermo Scientific), and, for each mutant strain, 20 μl (5 ng/μl) of DNA solution was submitted for NGS sequencing analysis. Each genomic DNA solution was used to create paired-end sequencing libraries and sequenced using 2×150 bp chemistry on a NEXTSEQ™ 500 System (Illumina Inc.) (as described in WO20123845). Sequence analysis was performed with the CLC Genomics Workbench version 11.0.1 (QIAGEN Inc.). Sequence analysis confirmed that: (1) the strains lacked the 70883 coding sequence but contained the 70883 homology flanks contained within plasmid D27XZX or D278ZE, (2) the strains contained a single copy of the amdS gene relative to an internal control single copy gene, (3) the strains lacked reads for the pGMER259 based Mad7 plasmid, and (4) that the strains also lacked reads from the plasmid D27XZX and D278ZE outside of the 5′ of the 70883 flanking sequence or the 3′ of the 70883 flanking sequence.

PCR from genomic DNA of strain O253QJ and O184PQ using primers 1232839 (SEQ ID NO:50) and 1232840 (SEQ ID NO:51) was used to confirm that the expected recombinant expression cassette containing an additional copy of the 108357 coding sequence had been integrated in the genome at the 70883 locus, see Table 2. The obtained 9.4 kb DNA fragment was confirmed to be the expected sequence by DNA sequencing with a Model 377 XL Automated DNA Sequencer (Applied Biosystems Inc.) using dye-terminated chemistry.

TABLE 2
PCR amplifications

| | | | Final |
|---|---|---|---|
| Component | Volume | | Concentration |
| 2X Phire Plant PCR Buffer | 10 | μl | 1x |
| 5 uM Primer F 1232839 | 0.2 | μl | 0.5 uM |
| 5 uM Primer R 1232840 | 0.2 | μl | 0.5 uM |
| Template DNA | 1 | μl | |
| Genomic DNA | 20-100 | ng/μl | |
| Phire Hot Start II DNA | 0.4 | μl | |
| Polymerase (2 U/ul) | | | |
| PCR grade water | 8.2 | μl | |
| Total reaction volume | 25 | μl | |

### 3-Step Cycle:

Step 1: Pre-denaturation: 98° C., 5 min
Step 2: Denaturation: 98° C., 10 sec.
Step 3: Annealing/Extension: 72° C., 4 min.
Step 4: Repeat steps 2-3, 40 times
Step 5: Final Extension: 72° C., 1 min.

### Example 3: Fed Batch Fermentation of the Generated Mutant Strains

The mutant T. reesei strains O16E5W, O253QJ, O184PQ and O1792Q were tested in 3-liter fed-batch fermentations to evaluate strain performance, recombinant enzyme activity levels and total protein expression levels.

The strains were each grown on a PDA plate for 5-9 days at 30° C. Three 500 ml shake flasks each containing 100 ml of shake flask medium for each strain were inoculated with two plugs from their respective PDA plate. The shake flasks were incubated at 26° C. for 48 hours on an orbital shaker at 250 rpm. The cultures were used as seeds for larger scale fermentation. A total of 160 ml of each seed culture was used to inoculate Applikon Biotechnology 3-liter glass jacketed fermentors containing 1.5 liters of fermentation batch medium. The fermenters were maintained at a temperature of 28° C. and pH was controlled using an Applikon control system to a set-point of 3.75+/−0.25. Air was added to the vessels at a rate of 2.5 L/min and the broths were agitated by Rushton impeller rotating at 1100 rpm. Fermentation feed medium composed of dextrose and phosphoric acid was dosed at a rate of 0 to 15 g/hour for a period of 163.75 hours based on a dissolved oxygen-controlled ramp. Daily samples of 1 ml were taken from each fermentor, centrifuged, and stored at −20° C.

Time point samples from the 3 L fermentations of strains O16E5W, O253QJ, O184PQ and O1792Q were submitted for Automated Total Protein assay, Automated pN-AMG assay, Automated β-mannosidase assay, and Automated MUL assay to evaluate whether the introduced gene was beneficial to Trichoderma reesei performance in a 3-liter fed-batch fermentation.

### Example 4: Increased Protein Production in Strains O184PQ and O1792Q

O184PQ and the control strain O1792Q were evaluated in lab-tanks under the current standard conditions in multiple independent tanks to investigate the effect of integration of plasmid D27XZX vs. the control plasmid D278ZE on their secreted enzyme activity. As can be seen in Table 3, compared to the control strain O1792Q, O184PQ showed 26% higher MUL titers, 39% higher AGU titers, 50% higher mannosidase titers, and 30% more total protein titers than control strain O1792Q at the end of the standard fermentation time course of 7 days. In conclusion, overexpression of the transcriptional regulatory polypeptide resulted in increased total host cell protein, and also in increased recombinant protein production, i.e. increased production of beta-mannosidase, CBH I and glucoamylase. This is in particular surprising since a drawback for utilizing endogenous transcription factors is that endogenous factors are typically under control of endogenous cellular regulation, and that it is challenging to identify conditions where this control matches the expectations of an industrial process, i.e. the end-product production level. Yet these challenges and drawbacks have been overcome by the present inventors as shown in the examples of this disclosure.

TABLE 3
Relative protein activity measurements in 3 L tank at the end of fermentation.

| | | | | Relative total |
|---|---|---|---|---|
| | | Relative | Relative | protein |
| | Relative MUL | glucoamylase | mannosidase | measured by |
| Strain & Plasmid | activity | activity | (dOD) activity | BCA |
| O184PQ | 126% | 139% | 150% | 130% |
| (D27XZX) | (p value 0.0179, | (p value 0.003, | (p value 0.0012, | (p value < 0.001, |
| 108357 (n = 3) | alpha 0.05) | alpha 0.05) | alpha 0.05) | alpha 0.05) |
| O1792Q | 100% | 100% | 100% | 100% |
| (D278ZE) | | | | |
| Control (n = 4) | | | | |

Activity is relative to activity of strain O1792Q (D278ZE), control host n=4. Comparisons analyzed by Dunnetts Method, control group was designated as O1792Q.

### Example 5: Decreased Culture Broth Viscosity and Increased Total Feed Usage for Strain O184PQ

A comparison of the total grams of feed used by strain O184PQ in the 7-day fermentation relative to the average of the total grams of feed used by strain O1792Q, demonstrates that strain O184PQ utilizes significantly increased amounts of total feed. As shown in Table 4, on average, O184PQ had a 24.5% increase in the relative amount of total feed used compared to O1792Q (Pvalue=<0.001, alpha 0.05, n=3 for each strain). The increased expression of the transcriptional regulatory polypeptide is therefore associated with lowered viscosity and with increased oxygen uptake rates.

TABLE 4
Relative total feed applied to fermentations
of O184PQ and O1972Q

| | Relative amount of total feed | |
|---|---|---|
| Strain | added during cultivation | p-value |
| O184PQ | 124.5% | <0.0001 |
| O1792Q | 100% | 1.0000 |

The feed dosing in fermentation is based on the dissolved oxygen measured in the tank. As can be seen in Table 4, the amount of feed, and therefore the amount of oxygen, applied to strain O184PQ was significantly greater than that applied to the control strain O1792Q, with about an 24.5% increase in feed and/or oxygen.

Since the fermentations were run using a dissolved oxygen ramp-based feed profile, the total feed has been taken as a proxy for culture broth viscosity. In general, during cell culture fermentations, reduced culture broth viscosities are related with increased oxygen mass transfer (A. Galaction et al., Biochemical Engineering Journal 20 (2004) 85-94). With decreased culture broth viscosity, the oxygen transfer into the bioreactor increases, more feed and oxygen can be added to the bioreactor, and the oxygen uptake rate of the culture broth is increased, as can be seen after overexpression of the transcriptional regulator in strain O184PQ. In contrast thereto, increased culture broth viscosities would lead to reduced oxygen transfer and therefore reduced total feed. Surprisingly, since the total feed and oxygen feed was increased for T. reesei strain O184PQ relative to the control strain O1792Q where the transcriptional regulatory polypeptide is not overexpressed, overexpression of the regulatory polypeptide results in reduced culture broth viscosity. Therefore, this would suggest that O184PQ has a preferential morphology which caused a decrease in viscosity compared to the control strain O1792Q.

### Example 6: Increased Protein Production in Strain O253QJ

Strain O253QJ and the control strain O16E5W were evaluated in lab-tanks under the current standard conditions in multiple independent tanks to investigate the effect of integration of plasmid D27XZX vs. the control plasmid D278ZE on their secreted enzyme activity. As shown in Table 5, compared to the control strain O16E5W, O253QJ showed 6% higher MUL titers, and 14% more total protein titers than control strain O16E5W at the end of the standard fermentation time course of 7 days. In conclusion, overexpression of the transcriptional regulatory polypeptide resulted in increased total protein secretion, and also in increased recombinant protein production/secretion.

TABLE 5
Relative protein activity measurements
in 3 L tank at the end of fermentation.

| | Relative MUL | Relative total protein |
|---|---|---|
| Strain & Plasmid | activity | measured by BCA |
| O253QJ | 106% | 114% |
| (D27XZX) | (p value 0.3, alpha | (p value 0.012, alpha |
| 108357 (n = 4) | 0.05) | 0.05) |
| O16E5W | 100% | 100% |
| (D278ZE) | | |
| Control (n = 3) | | |

Activity is relative to the activity of strain 16E5W (D278ZE), control host n=3. Comparisons analyzed by Dunnetts Method, control group was designated as O16E5W.

### Example 7: Decreased Culture Broth Viscosity and Increased Total Feed Usage for Strain O253QJ

A comparison of the total grams of feed used by strain O253QJ in the 7-day fermentation relative to the average of the total grams of feed used by strain O16E5W, demonstrates that strain O253QJ utilizes significantly increased amounts of total feed. As shown in Table 6, on average, O253QJ had a 5.9% increase in the relative amount of total feed used compared to O16E5W (Pvalue=<0.0024, alpha 0.05, n=3 for each strain). The increased expression of the transcriptional regulatory polypeptide in O253QJ is therefore associated with lowered viscosity and with increased oxygen uptake rates.

TABLE 6
Relative total feed applied to fermentations
of O253QJ and O16E5W

| | Relative amount of total feed | |
|---|---|---|
| Strain | added during cultivation | p-value |
| O253QJ | 105.9% | <0.0024 |
| O16E5W | 100% | 1.0000 |

The feed dosing in fermentation is based on the dissolved oxygen measured in the tank. As can be seen in Table 6, the amount of feed, and therefore the amount of oxygen, applied to strain O253QJ was significantly greater than that applied to the control strain O16E5W, with about an 5.9% increase in feed and/or oxygen.

Since the fermentations were run using a dissolved oxygen ramp-based feed profile, the total feed has been taken as a proxy for culture broth viscosity. In general, during cell culture fermentations, reduced culture broth viscosities are related with increased oxygen mass transfer (A. Galaction et al., Biochemical Engineering Journal 20 (2004) 85-94). With decreased culture broth viscosity, the oxygen transfer into the bioreactor increases, more feed and oxygen can be added to the bioreactor, and the oxygen uptake rate of the culture broth is increased, as can be seen after overexpression of the transcriptional regulator in strain O253QJ. In contrast thereto, increased culture broth viscosities would lead to reduced oxygen transfer and therefore reduced total feed. Surprisingly, since the total feed and oxygen feed was increased for T. reesei strain O253QJ relative to the control strain O16E5W where the transcriptional regulatory polypeptide is not overexpressed, overexpression of the regulatory polypeptide results in reduced culture broth viscosity. Therefore, this would suggest that O253QJ has a preferential morphology which caused a decrease in viscosity compared to the control strain O16E5W.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention is further defined by the following numbered paragraphs:
1. A fungal host cell comprising in its genome at least one first heterologous promoter operably linked to a first polynucleotide encoding a fungal transcriptional regulator polypeptide, or variant thereof, comprising or consisting of an amino acid sequence having a sequence identity of at least 60% to SEQ ID NO:24.
2. The fungal host cell of paragraph 1, wherein the transcriptional regulator polypeptide or variant thereof is heterologous to the recombinant host cell.
3. The fungal host cell of paragraph 1, wherein the transcriptional regulator polypeptide or variant thereof is endogenous to the recombinant host cell.
4. The fungal host cell according to any one of the preceding paragraphs, wherein the transcriptional regulator polypeptide, or variant thereof, comprises at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:79 and/or SEQ ID NO:80.
5. The fungal host cell according to any one of the preceding paragraphs, wherein the transcriptional regulator polypeptide, or variant thereof, comprises at least one DNA-binding motif comprising or consisting of an amino acid sequence corresponding to amino acids 257-281 of SEQ ID NO:24.
6. The fungal host cell according to any one of the preceding paragraphs, wherein the transcriptional regulator polypeptide, or variant thereof, comprises at least one DNA-binding motif comprising or consisting of an amino acid sequence corresponding to amino acids 286-311 of SEQ ID NO:24.
7. The fungal host cell according to any one of the preceding paragraphs, wherein the transcriptional regulator polypeptide, or variant thereof, comprises at least one DNA-binding motif comprising or consisting of an amino acid sequence corresponding to amino acids 257-281 of SEQ ID NO:24 and at least one DNA-binding motif comprising or consisting of an amino acid sequence corresponding to amino acids 286-311 of SEQ ID NO:24.
8. The fungal host cell according to any one of the preceding paragraphs, wherein the transcriptional regulator polypeptide, or variant thereof, comprises at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:79; and at least one DNA-binding motif comprising or consisting of an amino acid sequence having a sequence identity of at least 80%, e.g., at least 85%, at least 90%, at least 91%, at least 92%, at least 93%, at least 94%, at least 95%, at least 96%, at least 97%, at least 98%, at least 99%, or 100% to SEQ ID NO:80.
9. The fungal host cell according to any one of the preceding paragraphs, wherein the at least one DNA-binding motif comprises of, consists essentially of, or consists of the polypeptide with the amino acid of SEQ ID NO:79 and the polypeptide of SEQ ID NO:80.
10. The fungal host cell according to any one of the preceding paragraphs, wherein the at least one DNA-binding motif comprises of, consists essentially of, or consists of the polypeptide with the amino acid of SEQ ID NO:79 and the polypeptide of SEQ ID NO:80, wherein one or both of said polypeptide sequences comprise at least one amino acid substitution, amino acid deletion and/or amino acid insertion. Preferably, the at least one amino acid substation is a conservative amino acid substitution.
11. The fungal host cell according to any one of the preceding paragraphs, wherein the at least one DNA-binding motif comprises an amino acid sequence selected from the list of “FzRyEHLKRH”, wherein y=N or Q, and z=R or K (amino acids corresponding to amino acids 268-277 of SEQ ID NO:24); and “RyDNLNxH” wherein x=N or A; and y=Q or S (amino acids corresponding to amino acids 300-307 of SEQ ID NO:24).
12. The fungal host cell according to any one of the preceding paragraphs, wherein the host cell comprises at least two copies, e.g., three, four, or five copies of the at least one first heterologous promoter operably linked to the first polynucleotide.
13. The fungal host cell according to any one of the preceding paragraphs, wherein the host cell comprises at least two copies of the first polynucleotide encoding the transcriptional regulator polypeptide, such as one native copy and one or more additional copies each operably linked to the first heterologous promoter.
14. The fungal host cell according to any one of the preceding paragraphs, wherein the transcriptional regulator polypeptide or variant thereof is a regulator of xylanase regulator 1 (xyr1) gene expression, and/or a regulator of cellobiohydrolase 1 (cbh1) gene expression, preferably a regulator of the xyr1 promoter and/or a regulator of the cbh1 promoter.
15. The fungal host cell according to any one of the preceding paragraphs, wherein the transcriptional regulator polypeptide or variant thereof is a regulator of the xyr1 promoter and/or of the cbh1 promoter of a Trichoderma host cell.
16. The fungal host cell according to any one of the preceding paragraphs, wherein the transcriptional regulator polypeptide or variant thereof is a regulator of the xyr1 promoter and/or of the cbh1 promoter of a Trichoderma reesei host cell.
17. The fungal host cell according to any one of the preceding paragraphs, wherein the transcriptional regulator polypeptide, or variant thereof, comprises or consists of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO:24, and/or wherein the transcriptional regulator polypeptide, or variant thereof, is encoded by a first polynucleotide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO:11.
18. The fungal host cell according to any one of the preceding paragraphs, wherein the transcriptional regulator polypeptide or variant thereof comprises, consists essentially of, or consists of SEQ ID NO: 24, and/or wherein the first polynucleotide comprises, consists essentially of, or consists of SEQ ID NO:11.
19. The fungal host cell according to any one of the preceding paragraphs, wherein the at least one first heterologous promoter operably linked to a first polynucleotide confers an increased level of the transcriptional regulator polypeptide, or variant thereof, to the host cell relative to an isogenic cell lacking said nucleic acid construct or expression vector.
20. The fungal host cell according to any one of the preceding paragraphs, wherein the fungal host cell comprises in its genome at least one second heterologous promoter operably linked to at least one second polynucleotide encoding at least one polypeptide of interest, preferably the at least one second heterologous promoter is chosen from a polynucleotide sequence comprising or consisting of a nucleic acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:42, SEQ ID NO:45 and SEQ ID NO:55.
21. The fungal host cell according to paragraph 20, wherein the at least one polypeptide of interest is secreted.
22. The fungal host cell according to any one of the preceding paragraphs, wherein the fungal host cell comprises in its genome at least two first polynucleotides encoding the transcriptional regulator polypeptide, or variant thereof, such as two first polynucleotides, three first polynucleotides, four first polynucleotides, or more than four first polynucleotides encoding the transcriptional regulator polypeptide, or variant thereof.
23. The fungal host cell according to any one of the preceding paragraphs, wherein the first heterologous promoter operably linked to the first polynucleotide of the nucleic acid construct or expression vector is endogenous to the host cell.
24. The fungal host cell according to any one of the preceding paragraphs, wherein the first heterologous promoter comprises or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO:3.
25. The fungal host cell according to any one of the preceding paragraphs, wherein the first heterologous promoter is a constitutive promoter, a semi-constitutive promoter, a synthetic promoter, and/or an inducible promoter.
26. The fungal host cell according to any one of the preceding paragraphs, wherein the first heterologous promoter comprises, consists essentially of, or consists of SEQ ID NO: 3.
27. The fungal host cell according to any one of paragraphs 1-22 or 24-26, wherein the first heterologous promoter is not native to the host cell, such as being heterologous to the host cell.
28. The fungal host cell according to any one of the preceding paragraphs, wherein the fungal host cell is a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus oryzae, Fusarium venenatum, and Trichoderma reesei cell; most preferably the filamentous fungal host cell is an Trichoderma reesei cell.
29. The fungal host cell according to any one of paragraphs 1-28, wherein the host cell is a Trichoderma host cell, more preferably a Trichoderma reesei host cell.
30. The fungal host cell according to any one of paragraphs 1-27, wherein the host cell is a yeast host cell; preferably the yeast host cell is selected from the group consisting of Candida, Hansenula, Kluyveromyces, Pichia (Komagataella), Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell, most preferably the yeast host cell is Pichia pastoris (Komagataella phaffii).
31. The fungal host cell according to any one of paragraphs 1-30, wherein the at least one protein of interest is an endogenous protein of the host cell.
32. The fungal host cell according to any one of paragraphs 1-31, wherein the at least one protein of interest comprises or consists of at least two, at least three or at least four endogenous proteins of the host cell.
33. The fungal host cell according to any one of paragraphs 1-32, wherein the at least one protein of interest is the sum of all host cell proteins, preferably the sum of all secreted host cell proteins.
34. The fungal host cell according to any one of paragraphs 1-33, wherein the at least one polypeptide of interest has no cellulase activity (EC 3.2.1.4).
35. The fungal host cell according to any one of paragraphs 1-34, wherein the at least one polypeptide of interest comprises a heme-containing polypeptide selected from the group consisting of a NADPH-cytochrome P450 oxidoreductase (EC 1.6.2.4); a cytochrome B (EC 1.10.2.2); a peroxidase (EC 1.11.1) such as a catalase (EC 1.11.1.6), a cytochrome-C peroxidase (EC 1.11.1.5) or peroxidases categorized as EC 1.11.1.7; a peroxygenase (EC 1.11.2), such as a haloperoxidase (EC 1.11.2.1); a plant peroxidase or a halo-peroxidase; a cytochrome P450 enzyme (EC 1.14.14.1), such as a P450 mono-oxygenase or a P450 di-oxygenase; a heme 35 oxygenase (EC 1.14.99.3); a ferredoxin reductase (EC 1.18.1.3); a cytochrome bd-I oxidase (Cytochrome-D; EC 7.1.1.7); and a cytochrome c-oxidase (cytochrome A; EC 7.1.1.9; former EC 1.9.3.1); an active or an inactivated heme-containing enzyme selected from a list of polypeptides with at least 80% sequence identity to the polypeptides with SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, and SEQ ID NO:97; and/or a brazzein, a casein, a patatin, an ovalbumin, an osteopontin, an ovotransferrin, an ovomucin, an ovomucoid, an ovostatin, a lactoferrin, an alpha-lactalbumin, a beta-lactalbumin, a glycomacropeptide, and/or a collagen.
36. The fungal host cell according to any one of paragraphs 1-34, wherein the at least one polypeptide of interest comprises a therapeutic polypeptide selected from the group consisting of an antibody, an antibody fragment, an antibody-based drug, a Fc fusion protein, an anticoagulant, a blood factor, a bone morphogenetic protein, an engineered protein scaffold, an enzyme, a growth factor, a blood clotting factor, a hormone, an interferon (such as an interferon alpha-2b), an interleukin, a lactoferrin, an alpha-lactalbumin, a beta-lactalbumin, an ovomucoid, an ovostatin, a cytokine, an obestatin, a human galactosidase (such as an human alpha-galactosidase A), a vaccine, a protein vaccine, and a thrombolytic.
37. The fungal host cell according to any one of paragraphs 1-33, wherein the at least one polypeptide of interest is selected from the group consisting of hydrolase, isomerase, ligase, lyase, lysozyme, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.
38. The fungal host cell according to any one of paragraphs 1-33, wherein the at least one polypeptide of interest is a glycosylase, preferably a glycosidase, more preferably an amylase, cellobiohydrolase or mannosidase.
39. The fungal host cell according to any one of paragraphs 1-33, wherein the at least one polypeptide of interest is a hydrolase, preferably a glycosylase, more preferably a glycosidase; most preferably an amyloglucosidase (EC 3.2.1.3), such as an amyloglucosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:76.
40. The fungal host cell according to any one of paragraphs 1-33, wherein the at least one polypeptide of interest is a hydrolase, preferably a glycosylase; more preferably a glycosidase; most preferably a beta-mannosidase (EC 3.2.1.25), such as a beta-mannosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:77.
41. The fungal host cell according to any one of paragraphs 1-33, wherein the at least one polypeptide of interest is a hydrolase; preferably a glycosylase; more preferably a glycosidase; more preferably a cellobiohydrolase I or a cellobiohydrolase II (EC 3.2.1.91), such as a cellobiohydrolase I comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:78.
42. The fungal host cell according to any one of paragraphs 1-33, wherein at least two polypeptides of interest are encoded by the fungal host cell, wherein the at least two polypeptides of interested are selected from the list consisting of a cellobiohydrolase I comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:78, a beta-mannosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:77, and an amyloglucosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:76.
43. The fungal host cell according to any one of paragraphs 1-33, wherein at least three polypeptides of interest are encoded by the fungal host cell, wherein the at least three polypeptides of interest comprise a cellobiohydrolase I comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:78, a beta-mannosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:77, and an amyloglucosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:76.
44. The fungal host cell according to any one of paragraphs 1-33, wherein the first polynucleotide encoding the fungal transcriptional regulator polypeptide or variant thereof comprises one or more mutations, preferably nucleotide substitutions, nucleotide deletions or nucleotide insertions.
45. The fungal host cell according to paragraph 44, wherein the one or more mutations are leading to a variant of the transcriptional regulator polypeptide of SEQ ID NO: 24, such as a variant comprising (i) one or more additional amino acids compared to SEQ ID NO: 24, (ii) at least one amino acid less compared to SEQ ID NO: 24, e.g. a total of 10 to 20 amino acids, (iii) or an amino acid substitution of at least one amino acid of SEQ ID NO: 24, such as a conservative substitution of one or more amino acids at a position corresponding to positions 257 to 281 of SEQ ID NO:24, and/or a conservative substitution of one or more amino acids at a position corresponding to positions 286-311 of SEQ ID NO:24.
46. The fungal host cell according to any one of paragraphs 1-45, wherein the transcriptional regulator polypeptide differs by up to 10 amino acids, e.g., 1, 2, 3, 4, 5, 6, 7, 8, 9, or 10, from the mature polypeptide of SEQ ID NO:24.
47. The fungal host cell according to any one of paragraphs 1-45, wherein the transcriptional regulator polypeptide preferably comprises, consists essentially of, or consists of the amino acid sequence of SEQ ID NO:24; or is a fragment thereof having transcriptional regulator activity.
48. The fungal host cell according to any one of paragraphs 1-47, wherein the first polynucleotide hybridizes under medium stringency conditions, medium-high stringency conditions, high stringency conditions, or very high stringency conditions with the full-length complement of the mature polypeptide coding sequence of SEQ ID NO:25 or the cDNA thereof.
49. The fungal host cell according to any one of paragraphs 1-48, wherein the first polynucleotide is having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO:25, or SEQ ID NO:26.
50. The fungal host cell according to any one of paragraphs 1-49, wherein the first polynucleotide encoding the transcriptional regulator polypeptide comprises, consists essentially of, or consists of nucleotides 1 to 1160 of SEQ ID NO:25, or nucleotides 1 to 1092 of SEQ ID NO:26.
51. The fungal host cell according to any one of paragraphs 1-49, wherein the fungal transcriptional regulator polypeptide is derived from a mature polypeptide of SEQ ID NO:24 by substitution, deletion or addition of one or several amino acids in the mature polypeptide of SEQ ID NO:24, preferably the at least one substitution is a conservative amino acid substitution.
52. A method for producing at least one polypeptide of interest, the method comprising:
   i) providing a fungal host cell according to any one of the preceding paragraphs,
   ii) cultivating said fungal host cell under conditions conducive for expression of the at least one polypeptide of interest; and, optionally
   iii) recovering the at least one polypeptide of interest.
53. A nucleic acid construct comprising at least one first heterologous promoter operably linked to a first polynucleotide encoding a fungal transcriptional regulator polypeptide, or variant thereof, comprising or consisting of an amino acid sequence having a sequence identity of at least 60% to SEQ ID NO:24.
54. The nucleic acid construct according to paragraph 53, the transcriptional regulator polypeptide or variant thereof is a regulator of xylanase regulator 1 (xyr1) gene expression, and/or a regulator of cellobiohydrolase 1 (cbh1) gene expression, preferably a regulator of the xyr1 promoter and/or a regulator of the cbh1 promoter.
55. The nucleic acid construct according to any one of paragraphs 53-54, wherein the transcriptional regulator polypeptide, or variant thereof, comprises or consists of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:24.
56. The nucleic acid construct according to any one of paragraphs 53-55, wherein the first polynucleotide comprises or consists of an polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:25 or SEQ ID NO:26.
57. The nucleic acid construct according to any one of paragraphs 53-56, wherein the transcriptional regulator polypeptide or variant thereof comprises, consists essentially of, or consists of SEQ ID NO: 24.
58. The nucleic acid construct according to any one of paragraphs 53-57, wherein the first heterologous promoter is a constitutive promoter, a semi-constitutive promoter, a synthetic promoter and/or an inducible promoter.
59. The nucleic acid construct according to any one of paragraphs 53-58, wherein the first heterologous promoter operably linked to the first polynucleotide of the nucleic acid construct or expression vector is endogenous to the host cell.
60. The nucleic acid construct according to any one of paragraphs 53-59, wherein the first heterologous promoter comprises or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO:3.
61. An expression vector comprising a nucleic acid construct according to any one of paragraphs 53-60.
62. A method for generating a recombinant fungal host cell with increased protein secretion relative to an isogenic cell, the method comprising:
   i) providing a fungal host cell secreting at least one protein,
   ii) providing the at least one nucleic acid construct according to any one of paragraphs 53-60 or the at least one expression vector according to paragraph 61, and
   iii) integrating the at least one nucleic acid construct or the at least one expression vector into the genome of the host cell, wherein the at least one nucleic acid construct or the at least one expression vector confers an increased level of the transcriptional regulator polypeptide, or variant thereof, to the recombinant host cell relative to an isogenic cell lacking said nucleic acid construct or expression vector.
63. The method according to paragraph 62, wherein the method results in a host cell according to any one of paragraphs 1 to 51.
64. The method according to paragraph 62, wherein the method comprises an additional step: iv) integrating into the genome of the host cell at least one second heterologous promoter operably linked to a second polynucleotide encoding a polypeptide of interest.
65. A method for aerobic cultivation of recombinant fungal host cells, the method comprising:
   i) providing a fungal host cell according to any one of paragraphs 1 to 51,
   ii) cultivating the mutated fungal host cell under aerobic conditions conducive for expression of the at least one polypeptide of interest,
   wherein the aerobic cultivation of the fungal host cells is characterized by the formation of a culture broth with an increased oxygen uptake rate and/or a reduced viscosity, relative to the oxygen uptake rate and/or viscosity of a culture broth generated by the cultivation of an isogenic fungal host cells lacking the at least one nucleic acid construct and/or the at least one expression vector, when cultivated under identical conditions.
66. The method according to paragraph 65, wherein the increased oxygen uptake rate is determined by measuring the dissolved oxygen in a cultivation system, preferably a bioreactor.
67. The method according to any one of paragraphs 65 to 66, wherein the oxygen uptake rate OUR is increased by at least 10%, such as at least 15%, at least 20%, at least 25%, at least 30%, at least 35%, at least 40%, at least 45%, or at least 50% relative to the OUR of an isogenic cell when cultivated under identical or similar conditions.
68. The method according to any one of paragraphs 65 to 67, wherein the increased oxygen uptake rate is determined by measuring the oxygen feed supplemented into the cultivation system or bioreactor during a predetermined duration.
69. The method according to any one of paragraphs 65 to 68, wherein the reduced viscosity is determined by measuring the total feed supplemented into the cultivation system or bioreactor during a predetermined duration.
70. The method according to any one of paragraphs 65 to 69, wherein the total feed supplemented to the aerobic cultivation process of the fungal host cell is increased by at least 10%, such as at least 15%, at least 20%, at least 25%, at least 30%, at least 35%, at least 40%, at least 45%, or at least 50% relative to the OUR of an isogenic cell when cultivated under identical or similar conditions.
71. The method according to any one of paragraphs 65 to 70, wherein the reduced viscosity and/or increased oxygen uptake rate is determined by a reduced amount of agitation to maintain a preselected dissolved oxygen content compared to isogenic fungal host cells.
72. The method according to any one of paragraphs 65 to 70, wherein the reduced viscosity and/or increased oxygen uptake rate is determined by maintenance of an increased dissolved oxygen content at a preselected amount of agitation, compared to the isogenic fungal host cells.
73. A method for producing at least one transcriptional regulator polypeptide, the method comprising:
   i) providing a fungal host cell according to any one of paragraphs 1 to 51,
   ii) cultivating said fungal host cell under conditions conducive for expression of the at least one transcriptional regulator; and
   iii) optionally, recovering the at least one transcriptional regulator.
74. Use of a transcriptional regulator polypeptide for in-vitro transcription regulation, wherein the transcriptional regulator polypeptide is expressed by a fungal cell according to any one of paragraphs 1 to 51, or wherein the transcriptional regulator polypeptide is produced by the method according to paragraph 73.
75. Use according to paragraph 74, wherein the transcriptional regulator is used to regulate the transcription of a xyr1 promoter.
76. Use according to paragraph 74, wherein the transcriptional regulator is used to regulate the transcription of a cbh1 promoter.
77. Use according to any one of paragraphs 74 to 76, wherein the transcriptional regulator is used to regulate gene transcription in a cell-free expression system.
78. Use according to any one of paragraphs 74 to 77, wherein the use of the transcriptional regulator results in increased in-vitro protein expression of at least one protein of interest.
79. Use according to any one of paragraphs 74 to 78, wherein the cell-free expression system comprises cellular components of a fungal host cell, such as a fungal cell lysate.
80. Use according to any one of paragraphs 74 to 79, wherein the cell-free expression system comprises cellular components of a Trichoderma host cell.
81. Use according to any one of paragraphs 74 to 80, wherein the cell-free expression system comprises cellular components of a Trichoderma reesei host cell.
82. Use according to any one of paragraphs 74 to 81, wherein the cellular components of the cell-free expression system comprise one or more of a ribosome, a polymerase, at least one genomic DNA or DNA template, ATP, a cofactor, nucleotides, amino acids, and a tRNA.
83. Use according to any one of paragraphs 78 to 82, wherein the at least one polypeptide of interest has no cellulase activity (EC 3.2.1.4).
84. Use according to any one of paragraphs 78 to 82, wherein the at least one polypeptide of interest comprises a heme-containing polypeptide selected from the group consisting of a NADPH-cytochrome P450 oxidoreductase (EC 1.6.2.4); a cytochrome B (EC 1.10.2.2); a peroxidase (EC 1.11.1) such as a catalase (EC 1.11.1.6), a cytochrome-C peroxidase (EC 1.11.1.5) or peroxidases categorized as EC 1.11.1.7; a peroxygenase (EC 1.11.2), such as a haloperoxidase (EC 1.11.2.1); a plant peroxidase or a halo-peroxidase; a cytochrome P450 enzyme (EC 1.14.14.1), such as a P450 mono-oxygenase or a P450 di-oxygenase; a heme 35 oxygenase (EC 1.14.99.3); a ferredoxin reductase (EC 1.18.1.3); a cytochrome bd-I oxidase (Cytochrome-D; EC 7.1.1.7); and a cytochrome c-oxidase (cytochrome A; EC 7.1.1.9; former EC 1.9.3.1); an active or an inactivated heme-containing enzyme selected from a list of polypeptides with at least 80% sequence identity to the polypeptides with SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, and SEQ ID NO:97; and/or a brazzein, a casein, a patatin, an ovalbumin, an osteopontin, an ovotransferrin, an ovomucin, an ovomucoid, an ovostatin, a lactoferrin, an alpha-lactalbumin, a beta-lactalbumin, a glycomacropeptide, and/or a collagen.
85. Use according to any one of paragraphs 78 to 82, wherein the at least one polypeptide of interest comprises a therapeutic polypeptide selected from the group consisting of an antibody, an antibody fragment, an antibody-based drug, a Fc fusion protein, an anticoagulant, a blood factor, a bone morphogenetic protein, an engineered protein scaffold, an enzyme, a growth factor, a blood clotting factor, a hormone, an interferon (such as an interferon alpha-2b), an interleukin, a lactoferrin, an alpha-lactalbumin, a beta-lactalbumin, an ovomucoid, an ovostatin, a cytokine, an obestatin, a human galactosidase (such as an human alpha-galactosidase A), a vaccine, a protein vaccine, and a thrombolytic.
86. Use according to any one of paragraphs 78 to 82, wherein the at least one polypeptide of interest is selected from the group consisting of hydrolase, isomerase, ligase, lyase, lysozyme, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.
87. Use according to any one of paragraphs 78 to 82, wherein the at least one polypeptide of interest is a glycosylase, preferably a glycosidase, more preferably an amylase, cellobiohydrolase or mannosidase.
88. Use according to any one of paragraphs 78 to 82, wherein the at least one polypeptide of interest is a hydrolase, preferably a glycosylase, more preferably a glycosidase; most preferably an amyloglucosidase (EC 3.2.1.3), such as an amyloglucosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:76.
89. Use according to any one of paragraphs 78 to 82, wherein the at least one polypeptide of interest is a hydrolase, preferably a glycosylase; more preferably a glycosidase; most preferably a beta-mannosidase (EC 3.2.1.25), such as a beta-mannosidase comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:77.
90. Use according to any one of paragraphs 78 to 82, wherein the at least one polypeptide of interest is a hydrolase; preferably a glycosylase; more preferably a glycosidase; more preferably a cellobiohydrolase I or a cellobiohydrolase II (EC 3.2.1.91), such as a cellobiohydrolase I comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to SEQ ID NO:78.
91. A method for producing fungal biomass, the method comprising:
   i) providing a fungal host cell according to any one of paragraphs 1 to 51,
   ii) cultivating said fungal host cell under conditions conducive for expression of the transcriptional regulator polypeptide; and optionally
   iii) recovering the fungal host cells.
92. The method according to paragraph 91, wherein the host cell does not express a heterologous polypeptide of interest.
93. The method according to paragraph 91, wherein the host cell expresses a heterologous polypeptide of interest.
94. The method according to paragraph 93, wherein the heterologous polypeptide of interest is separated from the fungal host cell.
95. The method according to any one of paragraphs 91 to 94, wherein the fungal biomass comprises or consists of the fungal host cell and/or fungal host cell debris.

1-17. (canceled)

18: A fungal host cell comprising in its genome at least one first heterologous promoter operably linked to a first polynucleotide encoding a fungal transcriptional regulator polypeptide, or variant thereof, comprising or consisting of an amino acid sequence having a sequence identity of at least 80% to SEQ ID NO: 24.

19: The fungal host cell according to claim 18, wherein the transcriptional regulator polypeptide, or variant thereof, is endogenous to the host cell.

20: The fungal host cell according to claim 18, wherein the transcriptional regulator polypeptide, or variant thereof, comprises at least one DNA-binding motif comprising or consisting of an amino acid sequence having a sequence identity of at least 80% to SEQ ID NO:79 or SEQ ID NO: 80.

21: The fungal host cell according to claim 18, wherein the transcriptional regulator polypeptide comprises or consists of an amino acid sequence of SEQ ID NO: 24.

22: The fungal host cell according to claim 18, comprising in its genome at least one second heterologous promoter operably linked to at least one second polynucleotide encoding at least one polypeptide of interest.

23: The fungal host cell according to claim 22, wherein the at least one polypeptide of interest is secreted.

24: The fungal host cell according to claim 18, wherein the first heterologous promoter operably linked to the first polynucleotide is endogenous to the host cell.

25: The fungal host cell according to claim 18, wherein the host cell is a filamentous fungal host cell.

26: The fungal host cell according to claim 25, wherein the filamentous fungal host cell is a Trichoderma reesei cell.

27: The fungal host cell according to claim 18, wherein the host cell is a yeast host cell.

28: The fungal host cell according to claim 18, wherein the at least one polypeptide of interest comprises a heme-containing polypeptide, a brazzein, a casein, a patatin, an ovalbumin, an osteopontin, an ovotransferrin, an ovomucin, an ovomucoid, an ovostatin, a lactoferrin, an alpha-lactalbumin, a beta-lactalbumin, a glycomacropeptide, a collagen, and/or a therapeutic polypeptide.

29: The fungal host cell according to claim 18, wherein the at least one polypeptide of interest is selected from the group consisting of a hydrolase, isomerase, ligase, lyase, lysozyme, oxidoreductase, or transferase.

30: The fungal host cell according to claim 18, wherein the at least one polypeptide of interest is a glycosylase.

31: The fungal host cell according to claim 30, wherein the comprises or consists of of an amino acid sequence having a sequence identity of at least 80% to SEQ ID NO: 76, SEQ ID NO: 77, or SEQ ID NO: 78.

32: A method for producing at least one polypeptide of interest, the method comprising:
i) providing a fungal host cell according to claim 18,
ii) cultivating said fungal host cell under conditions conducive for expression of the at least one polypeptide of interest; and
iii) optionally, recovering the at least one polypeptide of interest.

33: A nucleic acid construct comprising at least one first heterologous promoter operably linked to a first polynucleotide encoding a fungal transcriptional regulator polypeptide, or variant thereof, comprising or consisting of an amino acid sequence having a sequence identity of at least 80% to SEQ ID NO:24.

34: An expression vector comprising a nucleic acid construct according to claim 33.

35: A method for generating a recombinant fungal host cell with increased protein secretion relative to an isogenic cell, the method comprising:
i) providing a fungal host cell secreting at least one protein of interest,
ii) providing the at least one nucleic acid construct of claim 33, and
iii) integrating the at least one nucleic acid construct or the at least one expression vector into the genome of the host cell,
wherein the at least one nucleic acid construct or the at least one expression vector confers an increased level of the transcriptional regulator polypeptide, or variant thereof, to the recombinant host cell relative to an isogenic cell lacking said nucleic acid construct or expression vector.

36: A method for aerobic cultivation of recombinant fungal host cells, the method comprising:
i) providing a recombinant fungal host cell according to claim 18,
ii) cultivating the recombinant fungal host cell under aerobic conditions conducive for expression of the at least one polypeptide of interest,
wherein the aerobic cultivation of the fungal host cells is characterized by the formation of a culture broth with an increased oxygen uptake rate and/or a reduced viscosity, relative to the oxygen uptake rate and/or viscosity of a culture broth generated by the cultivation of an isogenic fungal host cell lacking the at least one nucleic acid construct and/or the at least one expression vector, when cultivated under identical conditions.

37: A method for producing fungal biomass, the method comprising:
i) providing a fungal host cell according to claim 18,
ii) cultivating said fungal host cell under conditions conducive for expression of the transcriptional regulator polypeptide; and optionally
iii) recovering the fungal host cells.


SEQUENCE LISTING (97 sequences)

SEQ ID NO: 1 - DNA, 454 bp, Artificial Sequence
  E.coli pUC19
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcgtttaaac

SEQ ID NO: 2 - DNA, 2060 bp, Trichoderma reesei
ttggccacctacactgctactactacactgctattacttggtagctcttgggcttgcaggctcttcggcggataaccccatgttctttggtccgactatcttgcatctggcgactgggtcccattgattaggcccaaaagtcgcacaaaaaccgactgtaagccgccaatcagcctctctcggggctggtccccgttatgcccttttctctgcctcggtcgtttgagcccgcgactcaagtcgggtgccagcaacttcagcttcgccagaacccgtcacccaggtcgtggaactctgcgatttgctgcgggctgccgttccatctcgataaccaccgacgacacgaccgcatgctgtatcacgaaagcttctaacatctctgggcatcaggtatcacaccgttccagcggcgtctgtttcacgtcggttctcctccgcctagccggaccttggttccattcttgtttcgaagccgctggcctggcatagcttctcggtgccgtgaccataccctgcgttcaacaaacgacctgcttttcttttctcttccccttctgctcgtcctgagaccagctgccgagttctgtgtcttacttggggagacgagagcacggaaaggcacgaggttttatacgctcgtttaattacttcttgcggatatttcttccgttgaaggggaaatttcgacgactcttctgcgactgccaggagctcccaccgcgattttactacacaatattccaggctgctcctaatcttcgatttcacagtattgttcgactggaacaacatctttcccttccaattcattccgatctgttcacactcttgttcggtatacatgtacgaaacctgaagtctgctgttgctgggaattggacctgcggctccggagagtagccatctcgagtcctgcctgaatcatcctccatgacatcaataacgcatccctctaatccgacgccgcccctccttcctgggcatcatcgagcttaccccaacccggcccgctgtgcctttgacctcagctggggccgtaaagagcccagcacagaggagtcagctggacggagtagggcatatccgtcgcctcctatgtccggttctccgcccctacctctgagatcagctcatgaggctggtagtagaggtgaagccccgagttattatgctccccggctcttggatggccttcgcggcggcccagcgcaggcgcctcccacgagcaatcgagaccagacttcgccaataacgagatcctatccgcccgagccagcaacgaggtcgccatattcgtaccccagacccgaagacgcaggaaggatttacgtctacccccctcagcaccatcatggaatgccccagggagcttcagcagcgccgtatctgaactcagcatcctcggaaccgtatcctgcgcctgaccgatcacaggcagccgacacccagtccttgacatctccgaaatcacagagaaagacgaaaggccacgttgcttcggcttgcgtaccctgcaagaaagctcatcttcggtaagttccccagcggttggtgagatgcgtgattacgaggttcaggggtagtactaaatagctaacgctcctttttcctagatgcgacggtacgttgtgattatcatgaactttgatcttctgaagcaaagctggcattgttgagctgctggccctttctctcgggcaaaacgcatccattagaagccgcaggtgcccaaggtccagcaggcgagagttgtggacgtcctggcggaagtcggttaagacgcaagagtcaacggatcgtgatggagacaaggggtcgccaggaatagaagaaaatttcgaacgaaccgaagagctaacaatcaatatatagcacaacggccctgttcccgatgtatcggcaatgggaaggaggagtcttgcgtggacgttcagcataagaaaaggggccgaccgcgattacgagacgaaagagacgcaaggttcgaacctaccaggtactcgcaccaccaagatgcgtctctcaggagaccactgagtatatttccatctggagctagt

SEQ ID NO: 3 - DNA, 982 bp, Trichoderma reesei
gtacgtcaatgtaacgtcaaagccgccctcccgtaacctcgcccgttgttgctccccccgattgcctcaatcacatagtacctacctatgcattatggcgcctcaacccacccccccagattgagagctaccttacatcaatatggccagcacctcttcggcgatacatactcgccaccccagccggggcgattgtgtgtactaggtaggctcgtactataccagcaggagaggtgctgcttggcaatcgtgctcagctgttaggttgtacttgtatggtacttgtaaggtggtcatgcagttgctaaggtacctagggagggattcaacgagccctgcttccaatgtccatctggataggatggcggctggcggggccgaagctgggaactcgccaacagtcatatgtaatagctcaagttgatgataccgttttgccaggattaggatgcgagaagcagcatgaatgtcgctcatccgatgccgcatcaccgttgtgtcagaaacgaccaagctaagcaactaaggtaccttaccgtccactatctcaggtaaccaggtactaccagctaccctacctgccgtgcctacctgctttagtattaatctttccacctccctcctcaatcttcttttccctcctctcctcttttttttttcttcctcctcttcttctccataaccattcctaacaacatcgacattctctcctaatcaccagcctcgcaaatcctcaggttagtattactactactacaatcatcaccacgatgctccgcccgacgatgcggcttctgttcgcctgcccctcctctcactcgtgcccttgacgagctaccccgccagactctcctgcgtcaccaatttttttccctatttacccctcctccctctctccctctcgtttcttcctaacaaacaaccaccaccaaaatctctttggaagctcacgactcacgcaagctcaattcgcagatacaaa

SEQ ID NO: 4 - DNA, 37 bp, Artificial Sequence
  synthetic linker
gcggccgcgatctccgcatctacgcgtactagttaat

SEQ ID NO: 5 - DNA, 239 bp, Trichoderma reesei
taagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcat

SEQ ID NO: 6 - DNA, 2718 bp, Aspergillus nidulans
tggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatgatacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggagtcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccaaggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatgaagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtaga

SEQ ID NO: 7 - DNA, 2036 bp, Trichoderma reesei
aggcagccagtcgattcttcttggaaacactcatctccaaccttggcggccttttttagccctcgaaggtgtacgccgtacatcttgttcagtggttgatctttggcgcaggcgctcaggtttattcggaaatttgggttgaaattacaaccctatgcaacagaagagcgatgtttcttgggtttcctttgcatcaacggtttgtgaaagggcccgaacagtacagtcatatgttgtcggcaggagccggatctgaatttgcgcaatttggtggcatttgagtcaacttcactattgcttgatatgactcattttcccctcaaatcttcatttttgtttgtttgtctatttccttgatatggtctatcagctgaccttgatttctccttttggttattagacaagaaggggcggccagcgagcggtatcatgtttcaaagaggttggcggaggccaaattgatcgaatcaagcgttttcttatcaatgatatctttgactgggatcaaaggaatctgcatacatcaaatcatgtttttgtttcccattcggtattcttcttgcttcttctggctgtctctgtcttttttgttgggaatggcttcgcgaagcttgagcggtgatggcggcagcccatggtatagggtggcagtttggtttatacaattgttttgtctattgtttgttgagcaaatcttggggttggagattgggaggatttgtgcttgacagagatatgactacctagatattagcgacctgtatagcgtatagcgatttgtttttatggttctgatatttaagatacaatgatctgaagagtatcccccccctgattatgatttggtaacactttgacgcttggaaggctttgcgtcctgatctacagctatgcggaacgcagaatctccgaagactcaaccatgagcggcgacacccatgcgttagtctagccctggaaccatgattgtgtttgtctttctaaaaggtatactctcttttgctctcttttgttgcctcaccaaacagttaccgatccttttctcgcaataggagcttttgggttcaacctgtgctgttaccgggtggtgtatcaggtaccgtctcagtcacgtgcagacgtgggatgttcgcgtaagcatgcaagtcctcagctctttcctcccaccgccgtcactttctgtttcctgaaactcatgcaatgagaaaccctcggacctcgtcattttactcttctcttgcatttcgatcgttgcgttatacttctggctgttgtgcgacgggttgaccttctattggcgttgtttgatgatggtcggtgactcgggagtgcacatgttttacgtggtgtctgagtgcgatctattactgcgttgatcgaccggatagaattgatttcgttgccaaaagaaaaaaacaaaaatggatgggatggacattgacagcgccccgagcgggacgaagaggaaggctgtggatgagattgagtctccaaagccagcacgaaggatcagggtatggcatctctacgtgcttctctcatccttataccgcttgtgaagtggcgtggagggtcaatcttactaactcagtccctgcaatcaggccctcgatcccgatgttgtcaacaagatcgctgctggagagatcattgttgcgccggtccatgcgctcaaggaactcattgagaacgcagtggatgcagggtcgacgtcactggagatcctcgtcaaagacggaggtctgaagttgcttcagattacggacaacggcggcggcatcgaggtactttgcttttacctaggtacaatctcccactatcaacacagcgactgacactcttgaagaaagacgacttggaaatcttgtgcgtgcggcatacgacgtccaaaatttccacgttcgaagacctatcgtctattgccacatatggattccgaggcgaggcgctggcaagcatcagccacatcgcccatttgaccgtcactacaaagaccaaagaatcatccgtcgcctggcgtgcgcattatctggacg

SEQ ID NO: 8 - DNA, 2242 bp, Artificial Sequence
  E.coli pUC19
gtttaaacggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 60 bp, Artificial Sequence
  primer NZGP_EFP1DCDXMW_fwd
actcacgcaagctcaattcgcagatacaaaatgcagcacgccgagccagagtaccggttc

SEQ ID NO: 10 - DNA, 60 bp, Artificial Sequence
  primer NZGP_EFP1DCDXMW_rev
cggtgcgtcaggctttcgccacggagcttatcagtagctgtctgatcgcttctcgggcgc

SEQ ID NO: 11 - DNA, 1160 bp, Trichoderma reesei
atgcagcacgccgagccagagtaccggttcatggatgatggcctcactttggccataccatgccctacaaccagcttctcttcggcttcatctaattcatcttcaccgtacgaggtcttcacacccatttctcgtcgttcaacgcctaacaatctcaggctggactttgacggctaccagtcatatggaggtcacggagacctcacaccgccttcggcgatgcataagtacatgtttggtcccgtgaaagcggagcacggcactcttcccccttcaacaccactgatgaggaaaatgagcgacggcatgccctacgaccacatcctcgacatgaacaacatgaccacacactcactgggctccctcactccttcgggctctctacctgtttaccctggtgcctctttgccacattccccctacagcatttctcccacacacagcatctccccctccgagatgggagacaatgcgtcgtcgtggtgcaacaacaacagccccatcttttccctgggacagaaggtgcactctccccaagatgtcgagtctctggagcttgggtactcccactcccactcccactccccgatgagacaatactaccttcacagactcccgccgactcccaacaggttgtcggctcagagggaggccatgatccgcgaggcacgtctcaagacgacggagcttcacagccagatgaaggtcccgcgaagggtgcccgacaagcacgacagctcatacgacgtggtacgcaaggccatgtgcaagtgtgactaccccggctgccagaaggccttcaggaggaatgagcacttgaagcgtcacaagcaaacgtgagtattgcctcgaacgtctacaaagtcccagaccgtacgaacgaattgctaacgcgtcttattaggttccacggcgagggccccaaccgattctcctgcgagttctgcggcaaggaccagttcaaccgtcaagacaacctcaacaaccaccgcaaactacatgctcgccccaacagccgcaaccgcggggtggaattcatcccagctgcggtgcctgtcatcgagcaagaggagcgaagccgcaagaggcgggcgcctcccaagtcgagactgaccgcaggaggagcgccagcgcccgagaagcgatcagacagctactga

SEQ ID NO: 12 - DNA, 452 bp, Artificial Sequence
  E.coli pUC19
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagctt

SEQ ID NO: 13 - DNA, 500 bp, Magnaporthe oryzae
tctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttc

SEQ ID NO: 14 - DNA, 91 bp, Artificial Sequence
  tRNAgly(GCC)1-6
ggcttgcttgtcaagcaatggcatcattggtctagtggtagaattcgtcgttgccatcgacgaggcccgtgttcgattcacggatgatgca

SEQ ID NO: 15 - DNA, 21 bp, Artificial Sequence
  E. rectale sgRNA
ggaatttctactcttgtagat

SEQ ID NO: 16 - DNA, 6 bp, Artificial Sequence
  Bgl II recognition sequence
agatct

SEQ ID NO: 17 - DNA, 215 bp, Magnaporthe oryzae
tttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaat

SEQ ID NO: 18 - DNA, 886 bp, Aspergillus nidulans
cgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcatcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcacc

SEQ ID NO: 19 - DNA, 3816 bp, Artificial Sequence
  MAD7 coding sequence
atgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgataggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctccgaagaagaagcgaaaggtgtga

SEQ ID NO: 20 - DNA, 405 bp, Aspergillus nidulans
gcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggat

SEQ ID NO: 21 - DNA, 2234 bp, Artificial Sequence
  E.coli pUC19
ggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 22 - DNA, 93 bp, Artificial Sequence
  oligo 1231115
ttcacggatgatgcaggaatttctactcttgtagatgcattgtcaaagcatcgcccatttttttggctcttgggttcgaactgcccaaggccc

SEQ ID NO: 23 - DNA, 21 bp, Trichoderma reesei
gcattgtcaaagcatcgccca

SEQ ID NO: 24 - protein, 363 aa, Trichoderma reesei
MQHAEPEYRFMDDGLTLAIPCPTTSFSSASSNSSSPYEVFTPISRRSTPNNLRLDFDGYQSYGGHGDLTPPSAMHKYMFGPVKAEHGTLPPSTPLMRKMSDGMPYDHILDMNNMTTHSLGSLTPSGSLPVYPGASLPHSPYSISPTHSISPSEMGDNASSWCNNNSPIFSLGQKVHSPQDVESLELGYSHSHSHSPMRQYYLHRLPPTPNRLSAQREAMIREARLKTTELHSQMKVPRRVPDKHDSSYDVVRKAMCKCDYPGCQKAFRRNEHLKRHKQTFHGEGPNRFSCEFCGKDQFNRQDNLNNHRKLHARPNSRNRGVEFIPAAVPVIEQEERSRKRRAPPKSRLTAGGAPAPEKRSDSY

SEQ ID NO: 25 - DNA, 1160 bp, Trichoderma reesei; identical to SEQ ID NO: 11

SEQ ID NO: 26 - DNA, 1092 bp, Artificial Sequence
  cDNA of gene encoded by 108357
atgcagcacgccgagccagagtaccggttcatggatgatggcctcactttggccataccatgccctacaaccagcttctcttcggcttcatctaattcatcttcaccgtacgaggtcttcacacccatttctcgtcgttcaacgcctaacaatctcaggctggactttgacggctaccagtcatatggaggtcacggagacctcacaccgccttcggcgatgcataagtacatgtttggtcccgtgaaagcggagcacggcactcttcccccttcaacaccactgatgaggaaaatgagcgacggcatgccctacgaccacatcctcgacatgaacaacatgaccacacactcactgggctccctcactccttcgggctctctacctgtttaccctggtgcctctttgccacattccccctacagcatttctcccacacacagcatctccccctccgagatgggagacaatgcgtcgtcgtggtgcaacaacaacagccccatcttttccctgggacagaaggtgcactctccccaagatgtcgagtctctggagcttgggtactcccactcccactcccactccccgatgagacaatactaccttcacagactcccgccgactcccaacaggttgtcggctcagagggaggccatgatccgcgaggcacgtctcaagacgacggagcttcacagccagatgaaggtcccgcgaagggtgcccgacaagcacgacagctcatacgacgtggtacgcaaggccatgtgcaagtgtgactaccccggctgccagaaggccttcaggaggaatgagcacttgaagcgtcacaagcaaacgttccacggcgagggccccaaccgattctcctgcgagttctgcggcaaggaccagttcaaccgtcaagacaacctcaacaaccaccgcaaactacatgctcgccccaacagccgcaaccgcggggtggaattcatcccagctgcggtgcctgtcatcgagcaagaggagcgaagccgcaagaggcgggcgcctcccaagtcgagactgaccgcaggaggagcgccagcgcccgagaagcgatcagacagctactga

SEQ ID NO: 27 - DNA, 700 bp, Trichoderma reesei
cagaaggaataccagaacacctgaacctgctggaaggcaccttttagtccaccaactttggaacgacgcagcctcttctctcaaagctcagatcatagacgcagccagcgacatgcagtgcaatgtcatggatgttttgtgggttacgttcgtccctacagagtcactggcaggctacatgcatgtcatggtcccatatcgcccattcgaccaccgtcagcccagtacttgctcgtgtcaatctccgtcagtcgcgtgtgatcagacattccaactggatctgggccagccgggcactctttgccttgtcctgtttgacaggttcaatttgctctgtatgccacttctgtccagtctgtttagtgagccagactgctgagacactctgaaccgaggagcaagcgcctccacagccaagaagttgaagaagacggcagacacccacacagtgaccacgaagctttgtcacgcagtattgatcccatcaaccactagcaaatccctagacaccccgaaaggacttcaacagtgacctagcagcaaagccgtgagtctcggacggcctcttgttcaactcaaccgaccttccaagctacaaaaacgacaaggttggatgtctgccgtttgctgcctcgccaccagctgactttaggcaaaacaggtcattgaatccagatcggagtcgacactcgcatccgtg

SEQ ID NO: 28 - DNA, 8 bp, Artificial Sequence
  Synthetic spacer
ttaattaa

SEQ ID NO: 29 - DNA, 988 bp, Trichoderma reesei
cgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcacc

SEQ ID NO: 30 - DNA, 1705 bp, Artificial Sequence
  variant Rc-899
atgtttcgacgggctcttttcctgtcctcttccgccttccttgctgtcaaagcccagcagatcggcacggtcagtccggagaaccatccgcccctggcatgggagcagtgcactgcccctgggagttgcacgactgtgaatggtgcggtcgtccttgatgcgaactggcgttgggtccacaatgttgggggatacaccaactgctacactggcaatacctgggacaccacgtactgccctgacgacgtgacctgcgcagagaattgtgcgctggatggcgcagattacgagggcacctacggcgtgaccacctcgggcagctccctgaagctcgatttcgtcaccgggtctaacgtcggatctcgtctctacctgttggagaatgattcgacctatcagatcttcaagcttctgaaccaggaattcacctttgacgtcgacgtttccaatcttccgtgcggattaaacggcgctctgtaccttgttaccatggctgctgacggcggggtgtctcagtacccgaataacaaggccggcgcagcgtatggaaccggttattgcgattcccagtgtccaagggacttgaagtttatcgatggccaggtatgtagagctgtaatcacccatgttgtgaaatcactctcctactgacatggtcgatttataggccaacgttgagggctggcagccgtcttcgaacaacgccaatacaggtattggcaaccatggctcctgctgtgcggagatggatatctgggaagccaacagcatctccaatgcggtgactccgcacccatgcgacacacccggccagacaatgtgcgaggggaacgactgtggtggcacgtattccaccaatcgctatgcaggcacctgcgatcctgacggctgcgacttcaacccctaccgcatgggcaaccattctttctacggccctggggagattgtcgatactacccagcccttcacggtcgtgacacagttccttaccgatgatggcacggatactggcactctcagcgagatcaaacgcttctacgtccaaaacgggaaagtcattcctcagccgaactccgacattgccggcgtgactggcaactcgatcaccagcgagttttgcgatgcccagaagacggctttcggcgacattaacaactttgatacacacggcggtctggccagtatgggagctgcgctgcagcagggtatggttctggtgatgagtctgtgggacggtaggtccttgggagacacccggacgttctatatcaaccagaactgccagaactgacgaattaaaacacttttagattacgcggcaaacatgctgtggttggacagcatttatccaacaaatgcatctgctagcactcctggtgctgctcgtggaacctgttcgacgagctccggtgtcccatcgcaagtcgagtcgcagagccccaacgcctacgtgacgtactccaacattaaagttggaccaatcaactcgaccttcaccacttcgggctcgaaccctggaggcggaacgaccactactacaacgactcagccgacaacaacaactaccacagcaggcaaccctggaggtacaggtgtggcccagcactacggacagtgtggcggtatcggatggacaggacctactacttgtgcatcgccttatacctgtcagaaattgaacgactggtactcgcagtgtttgtaa

SEQ ID NO: 31 - DNA, 238 bp, Trichoderma reesei
aagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcat

SEQ ID NO: 32 - DNA, 6 bp, Artificial Sequence
  Synthetic spacer
agcgct

SEQ ID NO: 33 - DNA, 700 bp, Trichoderma reesei
ggttgaagggcttgtgttctttggctctgtagaggctctagggggttggattgggctgactatggtctcccctttgtatgctacacactgatcagacgatcgtagtcagttgagagatgagcttcattggtcgatatgagtgctttgagactggacacttgcgtgactgcatgttccgtttcttgtgataattgccgtgaagatgtgtctgctggtatgacgacaaagtagcatcagcagcaaagtatggcgaggactactaggcccaagatattgtcggcttcgtcaaagtgagaaattttagggtctccattgattacattcatccgtttcagaagcacgatatagaggagtagcaccatcaggtcggaaaggtcgtcaacagcttgaacaagtcatactaaggtatgttagtgtggtcgtggttgtcggtatcagtaggtatcattggagtatgtgttatatacagattaatgggaccaatgcatcacgcatcaggacatgccctggcaatcctccctcttacaaatcgcgatgaggcttgggtttgggggtaatagtagcggtcgtaaaggtggtggtgccagtagcgcacacgcagccctggccggaaatgctggtaatgggaggagatttgcaggtgggctcggtggtaaaggaggcgtaggtgctggtgggctcaaccgtgggctcaaccagc

SEQ ID NO: 34 - DNA, 700 bp, Trichoderma reesei
tcgcttaggcccttaagcttaggccggcttgcttactattaacctctcataaacgctactgcaatgattggaaacttcttatagtagaatgaggcaataagacgcatctcaggtcacatatagtcttatgtttgaaacccctcactactgccatttatcttgtggaaatatctattatttcagtctatacgtaatgaaggcacttttcaggatctcttccctaagcttgtataagcaggtttgttgccgtaaccattctgtctcctcgcctaatacctgtgaagcacagaatacgtttattctataagagacgtcttaccttccatcgagattgaaagcttaaaccgtctacaacggatgccctcatcatgacccgtctaactcgaacatctgccacattagtctcgggtaacaggaggagtaacacgaccagtgtaacacgttaagcatacaattgaacgagaatggtgaggactgagataaaagaattctgttaaggatctaaaattatagtgcatacaaggtagatgttagtaggtggtttcagttttcctttcctttacgttggtatagagcagcgttcaccaaatgttagcagagttctatctatgtcgtatccattctgccttatatctctcaagggcgccgagctcatcctacgaagctctcaggccatcgtaggaaatacaggatagacact

SEQ ID NO: 35 - DNA, 8 bp, Artificial Sequence
  Synthetic spacer
ttaattaa

SEQ ID NO: 36 - DNA, 988 bp, Trichoderma reesei; identical to SEQ ID NO: 29

SEQ ID NO: 37 - DNA, 1705 bp, Artificial Sequence; identical to SEQ ID NO: 30
  variant Rc-899

SEQ ID NO: 38 - DNA, 238 bp, Trichoderma reesei; identical to SEQ ID NO: 31

SEQ ID NO: 39 - DNA, 6 bp, Artificial Sequence
  Synthetic spacer
gctagc

SEQ ID NO: 40 - DNA, 700 bp, Trichoderma reesei
cacaatgtcgagtgtctattagacatactccgagaataaagtcaactgtgtctgtgatctaaagatcgattcggcagtcgagtagcgtataacaactccgagtaccagcaaaagcacgtcgtgacaggagcagggctttgccaactgcgcaaccttgcttgaatgaggatacacggggtgcaacatggctgtactgatccatcgcaaccaaaatttctgtttatagatcaagctggtagattccaattactccacctcttgcgcttctccatgacatgtaagtgcacgtggaaaccatacccaaattgcctacagctgcggagcatgagcctatggcgatcagtctggtcatgttaaccagcctgtgctctgacgttaatgcagaatagaaagccgcggttgcaatgcaaatgatgatgcctttgcagaaatggcttgctcgctgactgataccagtaacaactttgcttggccgtctagcgctgttgattgtattcatcacaacctcgtctccctcctttgggttgagctctttggatggctttccaaacgttaatagcgcgtttttctccacaaagtattcgtatggacgcgcttttgcgtgtattgcgtgagctaccagcagcccaattggcgaagtcttgagccgcatcgcatagaataattgattgcgcatttgatgcgatttttgagcggctgt

SEQ ID NO: 41 - DNA, 1522 bp, Trichoderma reesei
ggtgaaacaccgcccccttcttgagagagcaacaacaatcattctgctgtcggcagaagagcagagacttgctgaccctagttaatgactacacagctcggaggtttgtgacatgtccatgattttgatacatggcggagagcaatgtggtggacgaaatcaatcaccatatggcgctatattggctgtttcaggtcctgtttcaagctgttcctacagctctttcttggtctacttgtggtcgcctgctacatcagttgatatacccggaattactgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaa

SEQ ID NO: 42 - DNA, 1000 bp, Trichoderma viride
acccgaacctaattttatacaacgactttgattcagtctacagtaatgggacgtccccatatacagttgcacgtagggcacaacggtagagtacgttgggtgaattcgatatgatacgaggataaccccctgaatgtagagtctcacggcaaactctgaccgcgcggtgcgacctcacaaaacaatacaaacggatggctaaaagtacatgagttaatgcctaaagatgtcatataccagcggctaataattgtacaattaagtggctaaacgtaccgtaatttgccaatgacttgtagggttgcagaagcaacagtacagccccacttccccacgtttgccctcttacacgcaggtctaacctcaactgatgatctcccatctaagttctcttgttgttgtttagtctaagaggcaagtgtttacttcaggattttgtaaggcgtagcatgtaagaaataaacagaaagcagacgccaagaagcgagtttctggatgaaggcgtttgagagaaccttgcagggagttgtctgacaatagaaaaacaatggattgtcgcttctactcaggtgtctgtaattaaatgttactccgtcctgtacaggcaaaaaatatagtcgaatctgcctaagatctcgggccttcgggcctttaagtctacaggtcagtttggttatatgggcatttttgggtgtggtagcattgagggaaccactgcttttgccaaggagctgaacgtatgctgtaggcaaagctctaggtgccactgcatttgtgtcgaacataatgtgatgcttgggcaggcataatagccgccaaagatagcctcattgagcggaagtcggcgaacaggtgaagagcagaatatcacatatatatatggcccaaacgccgtgtccccttctccctttccccatctactcatcaactcagatcctccagaagacttgtacatcatcttttggggcatagcattctagtcgactacggactgcgcacc

SEQ ID NO: 43 - DNA, 1851 bp, Penicillium oxalicum
atgcgtctcactctattatcaggtgtagccggcgttctctgcgcaggacagctgacggcggcgcgtcctgatcccaagggtgggaatctgacgccgttcatccacaaagagggcgagcggtcgctccaaggcatcttggacaatctcggtgggcgaggtaagaaaacacccggcactgccgcagggttgtttattgccagtccaaacacagagaatccaaactattattatacatggactcgtgactcagctttgactgccaagtgcttgatcgacctgttcgaagactctcgggcagtctttccaattgaccgcaaatacttggaaacaggaattcgggactacgtgtcgtcccaagcaatcctccagagtgtgtctaatccttctggaaccctgaaggatggctctggtctgggtgaacccaagtttgagattgacctgaatcccttttcgggtgcctggggtcggcctcagcgggatggcccagcgctgcgagcgaccgctatgatcacctacgccaactacctgatatcccatggtcagaaatcggatgtgtcacaggtcatgtggccgattattgccaatgatctagcatatgttggtcaatactggaataataccggatttgacctgtgggaagaggtggatgggtcaagctttttcacgattgcggtccagcaccgagcccttgttgaaggctcgcaactggcgaaaaagctcggcaagtcctgcgatgcctgtgattctcagcctccccagatattgtgtttcctgcagagtttctggaacggaaagtacatcacctccaacatcaacacgcaagcaagccgctctggtatcgacctggactctgtcctgggaagcattcatacctttgatcccgaagcagcctgtgacgatgcaactttccagccttgttctgcccgcgctctggcgaaccacaaggtctatgtggattccttccgctctatctacaagattaatgcgggtcttgcagagggatcggctgccaacgttggccgctaccccgaggatgtttaccaaggaggcaatccatggtatctcgccaccctaggcgcatctgaattgctttacgacgccttgtaccagtgggacagacttggcaaacttgaagtctcggagacctcgttgtcattcttcaaagactttgacgcgaccgtgaaaattggctcgtactcgaggaacagcaagacctacaagaaattgacccagtccatcaagtcgtacgcggacgggttcatccagttagtgcagcagtacactccttctaatggatctctggccgagcaatacgatcgcaatacggctgctcctctctctgcaaacgatctgacttggtcatttgcctctttcttgacggctacgcaacgccgcgatgccgtggttcctccctcctggggcgcaaagtcggcaaacaaagtcccaaccacttgttcagcctcccctgttgtgggtacttataaggcgcccacggcaactttctcatccaagactaagtgcgtccccgctaaagatattgtgcctatcacgttctacctgattgagaacacttactatggagagaacgtcttcatgagtggcaacattactgcgctgggtaactgggacgccaagaaaggcttcccactcaccgcaaacctctacacgcaagatcaaaacttgtggttcgccagtgtcgagttcatcccagcaggcacaccctttgagtacaagtactacaaggtcgagcccaatggcgatattacttgggagaagggtcccaaccgggtgttcgtcgctcccacgggatgcccagttcagcctcactccaacgacgtgtggcagttttga

SEQ ID NO: 44 - DNA, 300 bp, Trichoderma viride
ggtactgtggcaaaagcttgaggtactgctggcttatggatgagttcatctcattatggactagatggaggatttactttgctgtatctacttctgaggcttccaatatatacggttatttcacctttgctggaatgctcgctagcttggcaagcacggctttcgagagacggactgattctctgctaactatgcattatataagactgaaatagacaaaaaaggaaaaaagttgccactcgaattatcttgacggtgttgattatatgtatggcattgtaagggtttttcattgatatt

SEQ ID NO: 45 - DNA, 1000 bp, Trichoderma harzianum
aggtagtcttgcagagcatagagcttcaaggtaaaactttcacgagattcactgatacgagcatgatggataacgtattccgtatatccacttataacaagtgttcaatcaaggaaatgctctgggcccttgccttcaattgggtctcatcgtgtgttttaggcactcaatgcaacgttatggaagactacaaaccggtggctaaaagtagatgggctattggctaaagatattataaactagcggctaaaaattcaccaattaagtggctaaacatttagcaactactaagaaggttacagaagaaaggataggccccacttccccacgttattctcttccgctcatctttaatctcactttgtccgagagccgttggttgcttttgcggcaatagtgtatgaagaggttccaataacaggaccagcaataagagagatgatgctgagtgaaatataaagcattgagtggattctgtgtcattatatccagttcgctatttgattggctagggagtttggtagccaatatgcaatgaagtagaaaataatttatgaggtctcgtatcgagacctgtgaatctttggtagaccgtgaccactggagtatgtactcgaaatataccggggtttctctgtacaggcaattgaaccaacttaatctgcctaaggaaatcgggccttcaaactagttagtgatcggaaggtattcggtgtggtaggagtgcattgttgcttttgccgtgcagcaaaatggatattgcaggtaaatgaccaggttgtagtgccgattgaccatgtgaattagaagcttaggcaggaataatagccggcgaagatagcctcattattctcatgtctgtggcaagaaaattatatatagtccaagctctttccaccatctccatctctctctccatctctccatcagctcggatactccaagagtcttacacatcatctttgtgtacatcatcttaagcaacgaaaggctgaacacc

SEQ ID NO: 46 - DNA, 3021 bp, Aspergillus niger
atgcgccacagcatcggattggcagcggccctactggcaccaaccctacctgtagcgttgggtcaatatattcgagacttaagcaccgagaaatggactctcagtagtcgagccttgaatcggacagtacctgctcaatttccatcgcaggttcacttagatctactaagggccggagtgattggtgagtactatttggaagtcaagtcctgtgagtatacaaccgctaacagcctcaatagatgatccgtaagtgacttcatctgccatggatgagaattgaatcgcactaaatattgctggagataccatggtttgaacgatttcaatcttcgctggatcgctgctgccaactggacttataccagtcaacccatcaaaggcctgtgagtcgctgtgaagtttgtgcaatgtcgttcgacaatactaagaaccaatagcctggacaattacgactcaacttggctcgtgtttgacggactggacactttcgcaacaatctcattctgtgggcagcaaatcgcatccacggacaatcagtttcgccagtatgcgttcgatgtatccaccgcactagggtcctgcaaaggagatcctgttctgagcatcaactttggaagcgcaccgaatattgttgatgctatcgcacaggactctaattcgcaaagtaagtttcagaggtgggggactgccgaagttgttacatgctaattgtatatagaatggcccgatgacgtccaactcacctacgagtacccaaatcggtggtttatgcgcaaagaacaatcggacttcggatgggattggggtccagcatttgcccctgcaggtccatggaagcctgcatatattgttcagctagacaagaaagaaagtgtctatgtcctgaacacggatttggatatataccgaaagggccaaattaactaccttccgccagaccagagccaaccttgggtcgtcaacgctagcattgacattttgggtccactacctaccaaaccaaccatgtcgattgaagtgcgcgatactcattctggcacgattcttacttcgcggactctgaacaatgtcagtgtggctggtaatgccataactggtgtcaccgttctcgacgggctgaccccgaaactgtggtggccgcaaggcctcggtgatcagaacctctacaatgtttctatcactgtccaaagtagaggaaaccagaccgtggccagtgtgaacaaacggacgggcttccgcaccatttttctcaaccagcgcaacattactgaagcacagcgtgcgcaaggaatcgcccctggagcaaactggcactttgaagtcaacggtcatgagttctacgcaaaaggatcgaaccttatcccaccagacagtttctggacccgtgttacagaagagaagatgtcacggctattcgatgcagtggtcgttggaaaccagaatatgctccgtgtctggtcctccggcgcgtacctgcatgactacatctatgatctggccgatgaaaagggcattctcttatggagcgagttcgagttcagtgacgctttatatccctccgacgacgctttcctcgagaacgttgctgctgagatagtatacaatgttcgacgagtgaaccaccatccctccttggctctatgggctggcggaaatgaaatcgaatccttgatgctcccacgtgtcaaagatgcagccccatcttcatattcctactatgtgggcgagtatgagaagatgtacattagcctcttcttgcctctggtctacgagaacacgcgttccatctcatactcccccagcagcacaaccgaaggctacctgtacattgacctttctgcccctgtcccaatggctgaacgttacgacaacactacctccggctcatactacggcgatacagaccactacgactacgacactagcgtggcgtttgactacggttcctatccggtaggccgctttgccaacgaattcggcttccacagcatgcccagcctccagacatggcaacaagctgtcgacactgaggatctttacttcaacagcagcgtcgtcatgctgcgcaaccaccacgatcccgcaggtggtctcatgacggacaactacgcgaactcggccactggcatgggcgaaatgaccatgggcgtggtaagctactatccgataccgagtaaatccgaccacatctccaacttcagcgcctggtgccatgccacccagctctttcaggcagacatgtacaaaagtcagatccagttctaccgtcgtggaagtggcatgcccgagcgccagcttggctccttgtattggcagctcgaagatatctggcaagcgccatcatgggcaggcattgagtacggtggtagatggaaggtccttcaccacgttatgagagatatctatcagcctgttattgtttcacctttttggaactatactaccggctcgttggatgtctatgttacttccgatctgtggagccctgcagcaggtactgtcgacttgacctggttggacctgtccggccgccctattgcgggtaacgcgggcacgccaaaatctgttccctttaccgtgggaggtctcaacagcactcgcatctatgggacgaatgtttcttctctgggcttgccggatactaaagatgctgttctgatcctctcgctctcggctcacggccgtcttccgaactcagaccggaccaccaacttgactcatgagaattacgctacgctttcttggcccaaggatttgaagattgttgacccgggacttaagataggacacagctcaaagaagacaaccgttacggtggaagctacatccggtgtttcattgtacacctggctcgactacccagagggtgtggtgggatactttgaagagaatgccttcgtcttagcaccaggcgagaagaaagagattagttttactgttctagaggacactactgacggggcttgggtccgtaacatcaccgtccagagtctctgggaccaaaaggttcgcggttga

SEQ ID NO: 47 - DNA, 300 bp, Trichoderma harzianum
ggtgctttgatggcggcttgagatgttaggttgtagatggattgtctcatcttcaactagatgtaggatttatttttattgtatctacttctgatgctagcaaagacggtcatttgatttggaactatggctgtttggtgttgcgatgaatgcttagctgctgggcaaaactggctttcgcaaagcatattgaccccttgctataaaagcattatatagaaagaacatggaactctttacaaaagtatatttactgagctactgattctcttttttgggccgcatgacaactttttgatc

SEQ ID NO: 48 - DNA, 1852 bp, Artificial Sequence
  artificial polynucleotide
caatattcatctctccatcgaagatggaaagaaaatactaattccgcaaaatgacaaatagaagtgacaacatcgtaattgaaaggatagtaaaaagcgatcatcaaaaggtcatgccagaaggtagcgtattatcatgaatgtttgaaaaagtgatatgcagtcaacgtaggtggtggaggtaggtggagaggaagaggacagacagtcaatgaccacgaggtcgcatgcgaatgggaagaaacacggccccaaagagaacgactaccgtccgcgatgaaaaaggaggggtattaatcattccaaaggatgggtgtacgagtgcagcaaataaagagatgcgtcattttcatcatcatcagaacaaccaacaaagagaaatacgaatcgcgcgaatcatcagttcaggaaagaactaacgaatcatttactattccttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatgctgggaacgcgaggtcagcggtgtggggcgaaagtggtacgacaaagcggaacgcgacataaccgcgtgttctgttggcgacagcagagcggtcgatgagcacgggcgaaatgtcgcgtccgtcgccgtctgtgacgatgaggactcacattagatattaggggataggaaatagaaagggctgggttgtcgttcaaggagatggattggacacgaagacaaaaatcagaaggtgccacgggcaaaggggattcttaaaaattcaaagtcagcttcattttccgtgtggcgcgtgcgcatgtttgcgtttgtgaggctgctcagggaccctttcccccaaaatttggaagcgaaatcgaccgctgggctgcccgtgaagccgtttaaatgaaggcc

SEQ ID NO: 49 - DNA, 1557 bp, Trichoderma reesei
gataacggaatagaagaaagaggaaattaaaaaaaaaaaaaaaacaaacatcccgttcataacccgtagaatcgccgctcttcgtgtatcccagtaccacggcaaaggtatttcatgatcgttcaatgttgatattgttcccgccagtatggctccacccccatctccgcgaatctcctcttctcgaacgcggtagtggcgcgccaattggtaatgacccatagggagacaaacagcataatagcaacagtggaaattagtggcgcaataattgagaacacagtgagaccatagctggcggcctggaaagcactgttggagaccaacttgtccgttgcgaggccaacttgcattgctgtcaagacgatgacaacgtagccgaggaccgtcacaagggacgcaaagttgtcgcggatgaggtctccgtagatggcatagccggcaatccgagagtagcctctcaacaggtggccttttcgaaaccggtaaaccttgttcagacgtcctagccgcagctcaccgtaccagtatcgaggattgacggcagaatagcagtggctctccaggatttgactggacaaaatcttccagtattcccaggtcacagtgtctggcagaagtcccttctcgcgtgcgagtcgaaagtcgctatagtgcgcaatgagagcacagtaggagaataggaacccgcgagcacattgttcaatctccacatgaattggatgactgctgggcagaatgtgctgcctccaaaatcctgcgtccaacagatactctggcaggggcttcagatgaatgcctctgggcccccagataagatgcagctctggattctcggttacgatgatatcgcgagagagcacgagttggtgatggaggggacgaggaggcataggtcggccgcaggcccataaccagtcttgcacagcattgatcttcctcacgaggagctcctgatgcagaaactcctccatgttgctgattgggttgagaatttcatcgctcctggatcgtatggttgctggcaagaccctgcttaaccgtgccgtgtcatggtcatctctggtggcttcgtcgctggcctgtctttgcaattcgacagcaaatggtggagatctctctatcgtgacagtcatggtagcgatagctaggtgtcgttgcacgcacataggccgaaatgcgaagtggaaagaatttcccggcgcggaatgaagtctcgtcattttgtactcgtactcgacacctccaccgaagtgttaagaatggatccacgatgccaaaaagcttgttcatttcggctagcccgtgatcctggcgcttctagggctgaaactgtgttgttaatgtattattggctgtgtaactgacttgaatggggaatgaggagcgcgatggattcgcttgcatgtcccctggccaagacgagccgctttggcggtttgtgattcgaaggtgtgtcagcggaggcgccagggcaacacgcactgagccagccaacatgcattgctgccgacatgaatagacacgcgccgagcagacataggagacgtgttgact

SEQ ID NO: 50 - DNA, 24 bp, Artificial Sequence
  Oligonucleotide F 1232839
taggattgaccgggcaggggatcg

SEQ ID NO: 51 - DNA, 26 bp, Artificial Sequence
  Oligonucleotide R 1232840
cgacgagtcggcacattgaagaagag

SEQ ID NO: 52 - DNA, 331 bp, Artificial Sequence
  E.coli pUC19
gccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgta

SEQ ID NO: 53 - DNA, 5725 bp, Artificial Sequence
  AMA1 sequence
ccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccgg

SEQ ID NO: 54 - DNA, 25 bp, Artificial Sequence
  synthetic linker
ggatcctctagagtcgacctgcagg

SEQ ID NO: 55 - DNA, 393 bp, Coprinus cinereus
ttcatttaaacggcttcacgggcagcccagcggtcgatttcgcttccaaattttgggggaaagggtccctgagcagcctcacaaacgcaaacatgcgcacgcgccacacggaaaatgaagctgactttgaatttttaagaatcccctttgcccgtggcaccttctgatttttgtcttcgtgtccaatccatctccttgaacgacaacccagccctttctatttcctatcccctaatatctaatgtgagtcctcatcgtcacagacggcgacggacgcgacatttcgcccgtgctcatcgaccgctctgctgtcgccaacagaacacgcggttatgtcgcgttccgctttgtcgtaccactttcgccccacaccgctgacctcgcgttcccagc

SEQ ID NO: 56 - DNA, 1032 bp, Artificial Sequence
  Hygromycin selection marker
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaggaatagtaaatga

SEQ ID NO: 57 - DNA, 423 bp, Coprinus cinereus
ttcgttagttctttcctgaactgatgattcgcgcgattcgtatttctctttgttggttgttctgatgatgatgaaaatgacgcatctctttatttgctgcactcgtacacccatcctttggaatgattaatacccctcctttttcatcgcggacggtagtcgttctctttggggccgtgtttcttcccattcgcatgcgacctcgtggtcattgactgtctgtcctcttcctctccacctacctccaccacctacgttgactgcatatcactttttcaaacattcatgataatacgctaccttctggcatgaccttttgatgatcgctttttactatcctttcaattacgatgttgtcacttctatttgtcattttgcggaattagtattttctttccatcttcgatggagagatgaatattg

SEQ ID NO: 58 - DNA, 19 bp, Artificial Sequence
  Synthesized in laboratory
cctgcaggcatgcaagctt

SEQ ID NO: 59 - DNA, 500 bp, Magnaporthe oryzae; identical to SEQ ID NO: 13

SEQ ID NO: 60 - DNA, 91 bp, Artificial Sequence; identical to SEQ ID NO: 14
  tRNAgly(GCC)1-6

SEQ ID NO: 61 - DNA, 21 bp, Artificial Sequence
  E. rectale sgRNA
ggaatttctactcttgtagat

SEQ ID NO: 62 - DNA, 216 bp, Magnaporthe oryzae
ctttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaat

SEQ ID NO: 63 - DNA, 886 bp, Aspergillus nidulans; identical to SEQ ID NO: 18

SEQ ID NO: 64 - DNA, 3816 bp, Artificial Sequence; identical to SEQ ID NO: 19
  Mad7 coding sequence

SEQ ID NO: 65 - DNA, 21 bp, Artificial Sequence
  SV40 NLS
ccgaagaagaagcgaaaggtg

SEQ ID NO: 66 - DNA, 405 bp, Aspergillus nidulans; identical to SEQ ID NO: 20

SEQ ID NO: 67 - DNA, 2471 bp, Artificial Sequence
  pUC19
ggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggc

SEQ ID NO: 68 - DNA, 81 bp, Artificial Sequence
  Oligonucleotide 1232807
gatgatgcaggaatttctactcttgtagatgtctccaaggatggcaagatttttttttggctcttgggttcgaactgccca

SEQ ID NO: 69 - DNA, 19 bp, Artificial Sequence
  protospacer sequence
gtctccaaggatggcaaga

SEQ ID NO: 70 - DNA, 30 bp, Artificial Sequence
  5′ homol. sequ. to pGMER263 at BglII site
gatgatgcaggaatttctactcttgtagat

SEQ ID NO: 71 - DNA, 32 bp, Artificial Sequence
  3′ homol. sequ.to pGMER263 at BglII site
tttttttttggctcttgggttcgaactgccca

SEQ ID NO: 72 - DNA, 81 bp, Artificial Sequence
  Oligonucleotide GMER263_fcy3
gatgatgcaggaatttctactcttgtagatgaccatggccgcagatctacgtttttttggctcttgggttcgaactgccca

SEQ ID NO: 73 - DNA, 21 bp, Artificial Sequence
  protospacer sequence
gaccatggccgcagatctacg

SEQ ID NO: 74 - DNA, 30 bp, Artificial Sequence; identical to SEQ ID NO: 70
  5′ homol. sequ. to pGMER263 at BglII site

SEQ ID NO: 75 - DNA, 32 bp, Artificial Sequence; identical to SEQ ID NO: 71
  3′ homol. sequ. to pGMER263 at BglII site

SEQ ID NO: 76 - protein, 616 aa, Penicillium oxalicum
MRLTLLSGVAGVLCAGQLTAARPDPKGGNLTPFIHKEGERSLQGILDNLGGRGKKTPGTAAGLFIASPNTENPNYYYTWTRDSALTAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 77 - protein, 931 aa, Aspergillus niger
MRHSIGLAAALLAPTLPVALGQYIRDLSTEKWTLSSRALNRTVPAQFPSQVHLDLLRAGVIGEYHGLNDFNLRWIAAANWTYTSQPIKGLLDNYDSTWLVFDGLDTFATISFCGQQIASTDNQFRQYAFDVSTALGSCKGDPVLSINFGSAPNIVDAIAQDSNSQKWPDDVQLTYEYPNRWFMRKEQSDFGWDWGPAFAPAGPWKPAYIVQLDKKESVYVLNTDLDIYRKGQINYLPPDQSQPWVVNASIDILGPLPTKPTMSIEVRDTHSGTILTSRTLNNVSVAGNAITGVTVLDGLTPKLWWPQGLGDQNLYNVSITVQSRGNQTVASVNKRTGFRTIFLNQRNITEAQRAQGIAPGANWHFEVNGHEFYAKGSNLIPPDSFWTRVTEEKMSRLFDAVVVGNQNMLRVWSSGAYLHDYIYDLADEKGILLWSEFEFSDALYPSDDAFLENVAAEIVYNVRRVNHHPSLALWAGGNEIESLMLPRVKDAAPSSYSYYVGEYEKMYISLFLPLVYENTRSISYSPSSTTEGYLYIDLSAPVPMAERYDNTTSGSYYGDTDHYDYDTSVAFDYGSYPVGRFANEFGFHSMPSLQTWQQAVDTEDLYFNSSVVMLRNHHDPAGGLMTDNYANSATGMGEMTMGVVSYYPIPSKSDHISNFSAWCHATQLFQADMYKSQIQFYRRGSGMPERQLGSLYWQLEDIWQAPSWAGIEYGGRWKVLHHVMRDIYQPVIVSPFWNYTTGSLDVYVTSDLWSPAAGTVDLTWLDLSGRPIAGNAGTPKSVPFTVGGLNSTRIYGTNVSSLGLPDTKDAVLILSLSAHGRLPNSDRTTNLTHENYATLSWPKDLKIVDPGLKIGHSSKKTTVTVEATSGVSLYTWLDYPEGVVGYFEENAFVLAPGEKKEISFTVLEDTTDGAWVRNITVQSLWDQKVRG

SEQ ID NO: 78 - protein, 521 aa, Artificial Sequence
  Cellobiohydrolase 1 variant Rc-899
MFRRALFLSSSAFLAVKAQQIGTVSPENHPPLAWEQCTAPGSCTTVNGAVVLDANWRWVHNVGGYTNCYTGNTWDTTYCPDDVTCAENCALDGADYEGTYGVTTSGSSLKLDFVTGSNVGSRLYLLENDSTYQIFKLLNQEFTFDVDVSNLPCGLNGALYLVTMAADGGVSQYPNNKAGAAYGTGYCDSQCPRDLKFIDGQANVEGWQPSSNNANTGIGNHGSCCAEMDIWEANSISNAVTPHPCDTPGQTMCEGNDCGGTYSTNRYAGTCDPDGCDFNPYRMGNHSFYGPGEIVDTTQPFTVVTQFLTDDGTDTGTLSEIKRFYVQNGKVIPQPNSDIAGVTGNSITSEFCDAQKTAFGDINNFDTHGGLASMGAALQQGMVLVMSLWDDYAANMLWLDSIYPTNASASTPGAARGTCSTSSGVPSQVESQSPNAYVTYSNIKVGPINSTFTTSGSNPGGGTTTTTTTQPTTTTTTAGNPGGTGVAQHYGQCGGIGWTGPTTCASPYTCQKLNDWYSQCL

SEQ ID NO: 79 - protein, 25 aa, Trichoderma reesei
KCDYPGCQKAFRRNEHLKRHKQTFH

SEQ ID NO: 80 - protein, 26 aa, Trichoderma reesei
NRFSCEFCGKDQFNRQDNLNNHRKLH

SEQ ID NO: 81 - protein, 363 aa, Coprinus cinereus
MKLSLLSTFAAVIIGALALPQGPGGGGSVTCPGGQSTSNSQCCVWFDVLDDLQTNFYQGSKCESPVRKILRIVFHDAIGFSPALTAAGQFGGGGADGSIIAHSNIELAFPANGGLTDTVEALRAVGINHGVSFGDLIQFATAVGMSNCPGSPRLEFLTGRSNSSQPSPPSLIPGPGNTVTAILDRMGDAGFSPDEVVDLLAAHSLASQEGLNSAIFRSPLDSTPQVFDTQFYIETLLKGTTQPGPSLGFAEELSPFPGEFRMRSDALLARDSRTACRWQSMTSSNEVMGQRYRAAMAKMSVLGFDRNALTDCSDVIPSAVSNNAAPVIPGGLTVDDIEVSCPSEPFPEIATASGPLPSLAPAP

SEQ ID NO: 82 - protein, 387 aa, Coprinus cinereus
MISTSKHLFVLLPLFLVSHLSLVLGFPAYASLGGLTERQVEEYTSKLPIVFPPPPPEPIKDPWLKLVNDRAHPWRPLRRGDVRGPCPGLNTLASHGYLPRDGVATPAQIITAVQEGFNMEYGIATFVTYAAHLVDGNPLTNLISIGGKTRKTGPDPPPPAIVGGLNTHAVFEGDASMTRGDFHLGDNFNFNQTLWEQFKDYSNRYGGGRYNLTAAAELRWARIQQSMATNGQFDFTSPRYFTAYAESVFPINFFTDGRLFTSNTTAPGPDMDSALSFFRDHRYPKDFHRAPVPSGARGLDVVAAAYPIQPGYNADGKVNNYVLDPTSADFTKFCLLYENFVLKTVKGLYPNPKGFLRKALETNLEYFYQSFPGSGGCPQVFPWGKSD

SEQ ID NO: 83 - protein, 313 aa, Humicola insolens
MMQFTTILSIGITVFGLGMYTTPLRLLSVTYVADWSANTGAFAAPQPVPEAYAVSDPEAHPDDFAGMDANQLQKRGFHDWEPPGPKDVRAPCPMLNTLANHGFLPHHGRDLTRKQVVDGLYNGLNINKTAASALFDFALMTSPKPNATTFSLNDLGRHNILEHDASLSRTDAYFGDVLAFNKTIFEETKRHWGKSPILDVTAAARARLGRIQTSKATNPEYFMSELGNIFTYGESVAYIMLIGDAKTGKANRRWVEYWFENERLPTHLGWRRPSKELTSDVLDAYISLIQNITLTLPGGTDPVKRRAASHFGW

SEQ ID NO: 84 - protein, 343 aa, Artificial Sequence
  CIP H55D
QGPGGGGSVTCPGGQSTSNSQCCVWFDVLDDLQTNFYQGSKCESPVRKILRIVFDDAIGFSPALTAAGQFGGGGADGSIIAHSNIELAFPANGGLTDTVEALRAVGINHGVSFGDLIQFATAVGMSNCPGSPRLEFLTGRSNSSQPSPPSLIPGPGNTVTAILDRMGDAGFSPDEVVDLLAAHSLASQEGLNSAIFRSPLDSTPQVFDTQFYIETLLKGTTQPGPSLGFAEELSPFPGEFRMRSDALLARDSRTACRWQSMTSSNEVMGQRYRAAMAKMSVLGFDRNALTDCSDVIPSAVSNNAAPVIPGGLTVDDIEVSCPSEPFPEIATASGPLPSLAPAP

SEQ ID NO: 85 - protein, 343 aa, Artificial Sequence
  CIP H55W
QGPGGGGSVTCPGGQSTSNSQCCVWFDVLDDLQTNFYQGSKCESPVRKILRIVFWDAIGFSPALTAAGQFGGGGADGSIIAHSNIELAFPANGGLTDTVEALRAVGINHGVSFGDLIQFATAVGMSNCPGSPRLEFLTGRSNSSQPSPPSLIPGPGNTVTAILDRMGDAGFSPDEVVDLLAAHSLASQEGLNSAIFRSPLDSTPQVFDTQFYIETLLKGTTQPGPSLGFAEELSPFPGEFRMRSDALLARDSRTACRWQSMTSSNEVMGQRYRAAMAKMSVLGFDRNALTDCSDVIPSAVSNNAAPVIPGGLTVDDIEVSCPSEPFPEIATASGPLPSLAPAP

SEQ ID NO: 86 - protein, 362 aa, Artificial Sequence
  WT392 I98W
FPAYASLGGLTERQVEEYTSKLPIVFPPPPPEPIKDPWLKLVNDRAHPWRPLRRGDVRGPCPGLNTLASHGYLPRDGVATPAQIITAVQEGFNMEYGWATFVTYAAHLVDGNPLTNLISIGGKTRKTGPDPPPPAIVGGLNTHAVFEGDASMTRGDFHLGDNFNFNQTLWEQFKDYSNRYGGGRYNLTAAAELRWARIQQSMATNGQFDFTSPRYFTAYAESVFPINFFTDGRLFTSNTTAPGPDMDSALSFFRDHRYPKDFHRAPVPSGARGLDVVAAAYPIQPGYNADGKVNNYVLDPTSADFTKFCLLYENFVLKTVKGLYPNPKGFLRKALETNLEYFYQSFPGSGGCPQVFPWGKSD

SEQ ID NO: 87 - protein, 362 aa, Artificial Sequence
  WT392 V102L
FPAYASLGGLTERQVEEYTSKLPIVFPPPPPEPIKDPWLKLVNDRAHPWRPLRRGDVRGPCPGLNTLASHGYLPRDGVATPAQIITAVQEGFNMEYGIATFLTYAAHLVDGNPLTNLISIGGKTRKTGPDPPPPAIVGGLNTHAVFEGDASMTRGDFHLGDNFNFNQTLWEQFKDYSNRYGGGRYNLTAAAELRWARIQQSMATNGQFDFTSPRYFTAYAESVFPINFFTDGRLFTSNTTAPGPDMDSALSFFRDHRYPKDFHRAPVPSGARGLDVVAAAYPIQPGYNADGKVNNYVLDPTSADFTKFCLLYENFVLKTVKGLYPNPKGFLRKALETNLEYFYQSFPGSGGCPQVFPWGKSD

SEQ ID NO: 88 - protein, 362 aa, Artificial Sequence
  WT392 V102W
FPAYASLGGLTERQVEEYTSKLPIVFPPPPPEPIKDPWLKLVNDRAHPWRPLRRGDVRGPCPGLNTLASHGYLPRDGVATPAQIITAVQEGFNMEYGIATFWTYAAHLVDGNPLTNLISIGGKTRKTGPDPPPPAIVGGLNTHAVFEGDASMTRGDFHLGDNFNFNQTLWEQFKDYSNRYGGGRYNLTAAAELRWARIQQSMATNGQFDFTSPRYFTAYAESVFPINFFTDGRLFTSNTTAPGPDMDSALSFFRDHRYPKDFHRAPVPSGARGLDVVAAAYPIQPGYNADGKVNNYVLDPTSADFTKFCLLYENFVLKTVKGLYPNPKGFLRKALETNLEYFYQSFPGSGGCPQVFPWGKSD

SEQ ID NO: 89 - protein, 362 aa, Artificial Sequence
  WT392 F224W
FPAYASLGGLTERQVEEYTSKLPIVFPPPPPEPIKDPWLKLVNDRAHPWRPLRRGDVRGPCPGLNTLASHGYLPRDGVATPAQIITAVQEGFNMEYGIATFVTYAAHLVDGNPLTNLISIGGKTRKTGPDPPPPAIVGGLNTHAVFEGDASMTRGDFHLGDNFNFNQTLWEQFKDYSNRYGGGRYNLTAAAELRWARIQQSMATNGQFDFTSPRYFTAYAESVWPINFFTDGRLFTSNTTAPGPDMDSALSFFRDHRYPKDFHRAPVPSGARGLDVVAAAYPIQPGYNADGKVNNYVLDPTSADFTKFCLLYENFVLKTVKGLYPNPKGFLRKALETNLEYFYQSFPGSGGCPQVFPWGKSD

SEQ ID NO: 90 - protein, 238 aa, Artificial Sequence
  Per27 C17H
GFHDWEPPGPKDVRAPHPMLNTLANHGFLPHHGRDLTRKQVVDGLYNGLNINKTAASALFDFALMTSPKPNATTFSLNDLGRHNILEHDASLSRTDAYFGDVLAFNKTIFEETKRHWGKSPILDVTAAARARLGRIQTSKATNPEYFMSELGNIFTYGESVAYIMLIGDAKTGKANRRWVEYWFENERLPTHLGWRRPSKELTSDVLDAYISLIQNITLTLPGGTDPVKRRAASHFGW

SEQ ID NO: 91 - protein, 238 aa, Artificial Sequence
  Per27 I154L
GFHDWEPPGPKDVRAPCPMLNTLANHGFLPHHGRDLTRKQVVDGLYNGLNINKTAASALFDFALMTSPKPNATTFSLNDLGRHNILEHDASLSRTDAYFGDVLAFNKTIFEETKRHWGKSPILDVTAAARARLGRIQTSKATNPEYFMSELGNLFTYGESVAYIMLIGDAKTGKANRRWVEYWFENERLPTHLGWRRPSKELTSDVLDAYISLIQNITLTLPGGTDPVKRRAASHFGW

SEQ ID NO: 92 - protein, 238 aa, Artificial Sequence
  Per27 L151C
GFHDWEPPGPKDVRAPCPMLNTLANHGFLPHHGRDLTRKQVVDGLYNGLNINKTAASALFDFALMTSPKPNATTFSLNDLGRHNILEHDASLSRTDAYFGDVLAFNKTIFEETKRHWGKSPILDVTAAARARLGRIQTSKATNPEYFMSECGNIFTYGESVAYIMLIGDAKTGKANRRWVEYWFENERLPTHLGWRRPSKELTSDVLDAYISLIQNITLTLPGGTDPVKRRAASHFGW

SEQ ID NO: 93 - protein, 238 aa, Artificial Sequence
  Per27 G158C
GFHDWEPPGPKDVRAPCPMLNTLANHGFLPHHGRDLTRKQVVDGLYNGLNINKTAASALFDFALMTSPKPNATTFSLNDLGRHNILEHDASLSRTDAYFGDVLAFNKTIFEETKRHWGKSPILDVTAAARARLGRIQTSKATNPEYFMSELGNIFTYCESVAYIMLIGDAKTGKANRRWVEYWFENERLPTHLGWRRPSKELTSDVLDAYISLIQNITLTLPGGTDPVKRRAASHFGW

SEQ ID NO: 94 - protein, 238 aa, Artificial Sequence
  Per27 G158W
GFHDWEPPGPKDVRAPCPMLNTLANHGFLPHHGRDLTRKQVVDGLYNGLNINKTAASALFDFALMTSPKPNATTFSLNDLGRHNILEHDASLSRTDAYFGDVLAFNKTIFEETKRHWGKSPILDVTAAARARLGRIQTSKATNPEYFMSELGNIFTYWESVAYIMLIGDAKTGKANRRWVEYWFENERLPTHLGWRRPSKELTSDVLDAYISLIQNITLTLPGGTDPVKRRAASHFGW

SEQ ID NO: 95 - protein, 238 aa, Artificial Sequence
  Per27 G158S
GFHDWEPPGPKDVRAPCPMLNTLANHGFLPHHGRDLTRKQVVDGLYNGLNINKTAASALFDFALMTSPKPNATTFSLNDLGRHNILEHDASLSRTDAYFGDVLAFNKTIFEETKRHWGKSPILDVTAAARARLGRIQTSKATNPEYFMSELGNIFTYSESVAYIMLIGDAKTGKANRRWVEYWFENERLPTHLGWRRPSKELTSDVLDAYISLIQNITLTLPGGTDPVKRRAASHFGW

SEQ ID NO: 96 - protein, 238 aa, Artificial Sequence
  Per27 G158A
GFHDWEPPGPKDVRAPCPMLNTLANHGFLPHHGRDLTRKQVVDGLYNGLNINKTAASALFDFALMTSPKPNATTFSLNDLGRHNILEHDASLSRTDAYFGDVLAFNKTIFEETKRHWGKSPILDVTAAARARLGRIQTSKATNPEYFMSELGNIFTYAESVAYIMLIGDAKTGKANRRWVEYWFENERLPTHLGWRRPSKELTSDVLDAYISLIQNITLTLPGGTDPVKRRAASHFGW

SEQ ID NO: 97 - protein, 238 aa, Artificial Sequence
  Per27 A162L
GFHDWEPPGPKDVRAPCPMLNTLANHGFLPHHGRDLTRKQVVDGLYNGLNINKTAASALFDFALMTSPKPNATTFSLNDLGRHNILEHDASLSRTDAYFGDVLAFNKTIFEETKRHWGKSPILDVTAAARARLGRIQTSKATNPEYFMSELGNIFTYGESVLYIMLIGDAKTGKANRRWVEYWFENERLPTHLGWRRPSKELTSDVLDAYISLIQNITLTLPGGTDPVKRRAASHFGW